(19) United States
(12) Patent Application Publication
(10) Pub. No.: US 2025/0041337 A1
(43) Pub. Date: Feb. 6, 2025

(54) FUSION PROTEIN

(71) Applicants: UNIVERSITÉ DE GENÈVE, Genève (CH); LES HÔPITAUX UNIVERSITAIRES DE GENÈVE, Genève (CH); THE TRUSTEES OF THE UNIVERSITY OF PENNSYLVANIA, Philadelphia, PA (US)

(72) Inventors: Denis MIGLIORINI, Geneva (CH); Jana DE SOSTOA, Lausanne (CH); Valerie DUTOIT, Copponex (FR); Pierre-Yves DIETRICH, Geneva (CH); Vincent ZOETE, Morges (CH); Justyna IWASZKIEWICZ, Lausanne (CH); Olivier MICHIELIN, Lausanne (CH); POSEY Avery, Philadelphia, PA (US)

(21) Appl. No.: 18/709,324

(22) PCT Filed: Nov. 11, 2022
(86) PCT No.: PCT/EP2022/081681

§ 371 Date: May 10, 2024

(30) Foreign Application Priority Data
Nov. 12, 2021 (EP) 21208014.7

(51) Int. Cl. A61K 35/17; A61K 39/00; A61P 35/04; C07K 14/53; C12N 9/64

(52) U.S. Cl. CPC A61K 35/17; A61K 39/4611; A61K 39/4631; A61K 39/464436; A61K 39/464454; A61P 35/04; C07K 14/53; C12N 9/6467; C07K 2319/43; C07K 2319/50; C12Y 304/21079

(57) ABSTRACT

The disclosure relates to a fusion protein comprising a colony stimulating factor 1 (CSF1) portion and a granzyme B portion, and polynucleotide sequence encoding the fusion protein. The disclosure also relates to a virus comprising the polynucleotide, and a T cell expressing the polynucleotide. The disclosure further relates to a method of treating a disease in an individual by administering the fusion protein, the polynucleotide, the oncolytic virus, or the T cell, and to a composition for use in method.

### FIELD OF THE DISCLOSURE

The disclosure relates to a fusion protein comprising a colony stimulating factor 1 (CSF1) portion and a granzyme B portion, and polynucleotide sequence encoding the fusion protein. The disclosure also relates to a virus comprising the polynucleotide, and a T cell expressing the polynucleotide. The disclosure further relates to a method of treating a disease in an individual by administering the fusion protein, the polynucleotide, the oncolytic virus, or the T cell, and to a composition for use in method.

### BACKGROUND

Cancer continues to represent a significant health problem worldwide, in spite of recent medical advances. Improved treatments are required, both for hematologic malignancies and solid tumours.

Acute myeloid leukaemia (AML) is an aggressive hematologic malignancy, with a five year survival rate of around 20%. The malignant cell in AML is the myeloblast, the precursor of myeloid white blood cells. Normally, a myeloblast matures into a monocyte or granulocyte (such as an eosinophil, basophil, or neutrophil). In AML, such maturation is disrupted, leading to proliferation of leukemic cells. Tyrosine kinase inhibitors and/or monoclonal antibodies may be used to treat AML in some individuals. However, chemotherapy is the mainstay of treatment for AML. As chemotherapy is associated with number side effects, and relapse is common, further treatments for AML are desired.

One aspect contributing to the successful treatment of hematologic malignancies (such as AML) and solid tumours is the ability to mount an effective immune response against cancer cells. However, the endogenous immune system is often non-reactive to cancer cells, seeing them as “self”. Even when endogenous immune cells are capable of recognising cancer cells, their anti-cancer responses may be suppressed by factors or cells in the microenvironment of solid tumours. Likewise, factors or cells in the tumour microenvironment may suppress the activity of immunotherapeutic agents, such as chimeric antigen receptor (CAR) T cells targeting a tumour antigen.

The microenvironment of solid tumours typically contains high numbers of tumour associated macrophages (TAMs). TAM populations tend to be heterogeneous, but include many immunosuppressive TAMs. These immunosuppressive TAMs express factors including the broad-acting immunosuppressive cytokines IL-10 and TGF-β. The presence of such factors in the tumour microenvironment dampens inflammatory immune responses and encourages the tolerisation of other immune cells, such as tumour-specific T cells. In addition, TAMs may secrete a range of chemokines that aid recruitment of suppressive cells, such as regulatory T cells (Tregs). TAMs may also interact physically with T cells to inhibit their proliferation and activation. Accordingly, mechanisms for reducing TAM-mediated immunosuppression may assisting the treatment of cancer by reducing inhibition upon anti-cancer immune responses. Therapeutics targeting TAMs are therefore also desired.

### SUMMARY OF THE DISCLOSURE

The present inventors have, for the first time, generated a fusion protein comprising a colony stimulating factor 1 (CSF1) portion and a granzyme B portion. The present inventors have demonstrated the fusion protein is cytotoxic to cells that express CSF1 receptor. As acute myeloid leukaemia (AML) cells express CSF1 receptor, they may be targeted by the fusion protein. Thus, the fusion protein may be used to treat AML. TAMs also express CSF1 receptor. Therefore, the fusion protein may be used to target TAMs, to reduce immunosuppression in the tumour microenvironment and thus promote anti-tumour immune responses. In this way, the fusion protein may be used to treat solid tumours. Similarly, the fusion protein may be used to treat inflammatory disease by targeting CSF1 receptor-expressing macrophages and/or monocytes involved in disease pathogenesis.

The disclosure therefore provides fusion protein comprising a colony stimulating factor 1 (CSF1) portion and a granzyme B portion. The disclosure further provides:
a polynucleotide sequence encoding the fusion protein of the disclosure;
a virus comprising the polynucleotide sequence of the disclosure;
a T cell comprising the polynucleotide sequence of the disclosure;
a method of treating a disease in an individual, comprising administering to the individual the fusion protein of the disclosure, the polynucleotide sequence of the disclosure, the oncolytic virus of the disclosure, or the T cell the disclosure; and
the fusion protein of the disclosure, the polynucleotide sequence of the disclosure, the oncolytic virus of the disclosure, or the T cell the disclosure for use in a method of treating a disease in an individual, the method comprising administering the fusion protein, the polynucleotide sequence, the oncolytic virus or the T cell to the individual.

### BRIEF DESCRIPTION OF THE FIGURES

FIG. 1: Simple fusion protein. The illustrated fusion protein consists of a leader sequence (SEQ ID NO: 12), FLAG-tag (SEQ ID NO: 10), GzmB (21-247) (SEQ ID NO: 7), a flexible linker ((GGGGS)₄) and CSF1 (33-181) (SEQ ID NO: 2). CSF1 (33-181) represents the receptor binding domain of CSF1. The fusion protein is shown bound to CSF1 receptor.

FIG. 2: Variation on fusion protein. The illustrated fusion protein consists of a leader sequence (SEQ ID NO: 12), FLAG-tag (SEQ ID NO: 10), GzmB (21-247) (SEQ ID NO: 7), a cleavable linker (SEQ ID NO: 14) and CSF1 (33-181) (SEQ ID NO: 2). The fusion protein is shown bound to CSF1 receptor.

FIG. 3: Modifications to facilitate endosomal release. The illustrated fusion protein consists of a leader sequence (SEQ ID NO: 12), FLAG-tag (SEQ ID NO: 10), GzmB (21-247) (SEQ ID NO: 7), a flexible linker ((GGGGS)₄), CSF1 (33-196) (SEQ ID NO: 3), and a fusogenic peptide (SEQ ID NO: 11). The fusion protein is shown bound to CSF1 receptor. The extended CSF1 portion (CSF1 (33-196)) contains double Cys (i.e. disulfide) bonds that may improve endosomal release. The fusogenic peptide forms an alpha helix, which may also improve endosomal release. Only one of (i) the extended CSF1 portion (CSF1 (33-196)) and (ii) the fusogenic peptide is needed to improve endosomal release, though both may be present in the fusion protein.

FIG. 4: Variation on fusion protein. The illustrated fusion protein consists of a leader sequence (SEQ ID NO: 12), FLAG-tag (SEQ ID NO: 10), GzmB (21-247) (SEQ ID NO: 7), a flexible linker ((GGGGS)₄) and C63S/M59R mutant CSF1 (33-181) (SEQ ID NO: 4). The fusion protein is shown bound to CSF1 receptor.

FIG. 5: Variation on fusion protein. Results of flow cytometry showing EK293 cell transfection with GzmB-CSF1-FL (SEQ ID NO: 15), GzmB-CSF1-FLFP (SEQ ID NO: 16), GzmB-CSF1-CL (SEQ ID NO: 17), GzmB-CSF1-FLFPlong (SEQ ID NO: 18), or GzmB-CSF1 (SEQ ID NO: 19).

FIG. 6: CSF1R expression of Mono mac 1 and Thp1 cells. Indicated cells were evaluated for CSF1R expression by flow cytometry. Cells were incubated with an APC-coupled anti-CSF1R antibody or its corresponding isotype control. Results of flow cytometry show that 36.4% of MonoMac1 cells and 88.2% of THP1 cells expressed CSF1R at a level above that of the isotype control.

FIG. 7: Cartoon showing method used to perform cytotoxicity assay. HEK293 were transfected with the different pTRPE-GzmB-CSF1 versions. After 72 h, supernatants (SNTs) were collected and incubated with A549 (5% CSF1R-positive cells), Thp1 (84% CSF1R-posivite cells), and Mono mac 1 (30% CSF1R-positive cells) for 72 h. Cellular viability was measured by flow cytometry after LIVE/DEAD staining.

FIG. 8: Results of a first cytotoxicity assay. Cytotoxicity of GzmB-CSF1 fusion proteins-containing supernatants of HEK293 transfected cells. A549, Mono mac 1, and Thp1 cells were incubated with non-concentrated supernatants for 72 hours. Cell viability was measured by flow cytometry after LIVE/DEAD staining. Mean values±SD are plotted (n=3).

FIG. 9: Results of a second cytotoxicity assay. Cytotoxicity of GzmB-CSF1 fusion proteins-containing supernatants of HEK293 transfected cells. A549, Mono mac 1, and Thp1 cells were incubated with non-concentrated (1×) or 10× concentrated (10×) supernatants for 72 hours. Cell viability was measured by flow cytometry after LIVE/DEAD staining. Mean values±SD are plotted (n=3).

FIG. 10: Plot representation of the cytotoxicity assay (FIG. 9) with non-concentrated supernatants.

FIG. 11: Plot representation of the cytotoxicity assay (FIG. 9) with 10× concentrated supernatants.

FIG. 12: Purification and cleavage of the GzmB-CSF1 fusion protein. GzmB-CSF1-CL fusion protein was digested with an Enterokinase enzyme to obtain an active GzmB-CSF1-CL protein. Enterokinase cleavage site is DYKDDDDK, which corresponds to the FLAG-tag sequence on the GzmB-CSF1-CL fusion protein. Western Blot (WB) was performed after digestion to detect the loss of FLAG-tag on digested GzmB-CSF1-CL fusion protein. Cleaved and uncleaved GzmB-CSF1-CL was used to compare cytotoxicity capacity and specificity on different hCSF1R-positive and negative cell lines (FIG. 13).

FIG. 13: Cytotoxic characterization of cleaved and uncleaved purified GzmB-CSF1-CL. The glioblastoma patient derived-cell line Ge518 and the lung cancer cell line A549 were transduced with a lentivirus to express the human CSF1R on the membrane (Ge518-hCSF1R and A549-hCSF1R, respectively). Cells were cocultured with different concentrations of the cleaved or uncleaved GzmB-CSF1-CL fusion protein, and the cytotoxicity was evaluated over time using the Incucyte machine. Data represent mean±SD of apoptotic index (cell death/confluence).

### BRIEF DESCRIPTION OF THE SEQUENCE LISTING

SEQ ID NO: 1—amino acid sequence of wild-type CSF1.
SEQ ID NO: 2—amino acid sequence of an exemplary CSF1 portion, CSF1 (33-181).
SEQ ID NO: 3—amino acid sequence of an exemplary CSF1 portion, CSF1 (33-196).
SEQ ID NO: 4—amino acid sequence of an exemplary CSF1 portion, C63S/M59R mutant CSF1 (33-181).
SEQ ID NO: 5—amino acid sequence of an exemplary CSF1 portion, C63S/M59R mutant CSF1 (33-196).
SEQ ID NO: 6—amino acid sequence of wild-type granzyme B.
SEQ ID NO: 7—amino acid sequence of an exemplary granzyme B portion, granzyme B (21-247).
SEQ ID NO: 8—amino acid sequence of an exemplary granzyme B portion, R221K mutant granzyme B (21-247).
SEQ ID NO: 9—amino acid sequence of an exemplary granzyme B portion, R116A/R120A/R122A/K241A/K242A/K245A/R246A mutant granzyme B (21-247).
SEQ ID NO: 10—amino acid sequence of an exemplary FLAG-tag.
SEQ ID NO: 11—amino acid sequence of an exemplary fusogenic peptide that forms an alpha helix motif.
SEQ ID NO: 12—amino acid sequence of an exemplary leader sequence.
SEQ ID NO: 13—amino acid sequence comprised in an exemplary linker.
SEQ ID NO: 14—amino acid sequence of an exemplary cleavable linker.
SEQ ID NO: 15—amino acid sequence of GzmB-CSF1-FL, a fusion protein of the Examples.
SEQ ID NO: 16—amino acid sequence of GzmB-CSF1-FLFP, a fusion protein of the Examples.
SEQ ID NO: 17—amino acid sequence of GzmB-CSF1-CL, a fusion protein of the Examples.
SEQ ID NO: 18—amino acid sequence of GzmB-CSF1-FLFPlong, a fusion protein of the Examples.
SEQ ID NO: 19—amino acid sequence of GzmB-CSF1, a fusion protein of the Examples.
SEQ ID NO: 20—amino acid sequence of GzmB-CSF1-FL minus FLAG-tag.
SEQ ID NO: 21—amino acid sequence of GzmB-CSF1-FLFP minus FLAG-tag.
SEQ ID NO: 22—amino acid sequence of GzmB-CSF1-CL minus FLAG-tag.
SEQ ID NO: 23—amino acid sequence of GzmB-CSF1-FLFPlong minus FLAG-tag.
SEQ ID NO: 24—amino acid sequence of GzmB-CSF1-FL minus leader.
SEQ ID NO: 25—amino acid sequence of GzmB-CSF1-FLFP minus leader.
SEQ ID NO: 26—amino acid sequence of GzmB-CSF1-CL minus leader.
SEQ ID NO: 27—amino acid sequence of GzmB-CSF1-FLFPlong minus leader.
SEQ ID NO: 28—amino acid sequence of GzmB-CSF1 minus leader.
SEQ ID NO: 29—amino acid sequence of GzmB-CSF1-FL minus leader and FLAG-tag.
SEQ ID NO: 30—amino acid sequence of GzmB-CSF1-FLFP minus leader and FLAG-tag.
SEQ ID NO: 31—amino acid sequence of GzmB-CSF1-CL minus leader and FLAG-tag.
SEQ ID NO: 32—amino acid sequence of GzmB-CSF1-FLFPlong minus leader and FLAG-tag.
SEQ ID NO: 33—amino acid sequence of an exemplary cleavable linker.
SEQ ID NO: 34—amino acid sequence of an exemplary cleavable linker.
SEQ ID NO: 35—amino acid sequence comprised in an exemplary cleavable linker.
SEQ ID NO: 36—amino acid sequence of GzmB-CSF1-CL containing alternative cleavable linker.
SEQ ID NO: 37—amino acid sequence of GzmB-CSF1-CL containing alternative cleavable linker, minus FLAG-tag.
SEQ ID NO: 38—amino acid sequence of GzmB-CSF1-CL containing alternative cleavable linker, minus leader.
SEQ ID NO: 39—amino acid sequence of GzmB-CSF1-CL containing alternative cleavable linker, minus leader and FLAG-tag.

### DETAILED DESCRIPTION

It is to be understood that different applications of the disclosed products and methods may be tailored to the specific needs in the art. It is also to be understood that the terminology used herein is for the purpose of describing particular aspects of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a fusion protein” includes “fusion proteins”, reference to “an oncolytic virus” includes two or more such oncolytic viruses, and the like.

In general, the term “comprising” is intended to mean including but not limited to. For example, the phrase “a fusion protein comprising a colony stimulating factor 1 (CSF1) portion and a granzyme B portion” should be interpreted to mean that the fusion protein includes a CSF1 portion and a granzyme B portion, but that the fusion protein may also include one or more other components.

In some aspects of the disclosure, the word “comprising” is replaced with the phrase “consisting of”. The term “consisting of” is intended to be limiting. For example, the phrase “a fusion protein consisting of a colony stimulating factor 1 (CSF1) portion and a granzyme B portion” should be understood to mean that the fusion protein includes only a CSF1 portion and a granzyme B portion, and no further components.

The terms “protein” and “polypeptide” are used interchangeably herein, and are intended to refer to a polymeric chain of amino acids of any length.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides/residues at positions within the sequence are then compared. When a position in the first sequence is occupied by the same nucleotide/residue as the corresponding position in the second sequence, then the nucleotides/residues are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm

### Fusion Protein

Disclosed herein is fusion protein comprising a colony stimulating factor 1 (CSF1) portion and a granzyme B portion. The fusion protein is associated with a number of advantages, which will become apparent in the following sections. In brief, though, the structure of the fusion protein confers it with the ability to have therapeutic effect in a number of conditions. The CSF1 portion of the fusion protein endows it with the ability to bind to CSF1 receptor. In this way, the fusion protein can be targeted to CSF1 receptor-expressing cells. The granzyme B portion of the fusion protein endows it with cytotoxic activity. In this way, the fusion protein can be used to kill cells to which it is targeted.

The targeted destruction of CSF1 receptor-expressing cells is of therapeutic benefit in a number of conditions. For example, acute myeloid leukaemia (AML) cells express CSF1 receptor, and so the fusion protein may be administered to an individual in order to treat AML. Monocytes and macrophages involved in the pathogenesis of inflammatory diseases also express CSF1 receptor, and so the fusion protein may be administered to an individual in order to treat an inflammatory disease. Furthermore, tumour associated macrophages (TAMs) express CSF1 receptor, and have an immunosuppressive effect on the microenvironment of solid tumours. The fusion protein may therefore be administered to an individual in order target TAMs for destruction and thereby reduce immunosuppression in the tumour microenvironment. In this way, the fusion protein may be used to treat a solid tumour.

### Fusion Protein

A fusion protein is a chimeric protein that may be created through the joining of two or more polynucleotide sequences each encoding a different protein. Translation of the resultant joined polynucleotide sequence generates a single protein that comprises the encoded parts of each different protein. In essence, each different protein is fused together in a single protein. The single protein, or “fusion protein”, typically has functional properties derived from each different protein.

The fusion protein of the present disclosure comprises a CSF1 portion and a granzyme B portion. The fusion protein may therefore be created by joining a polynucleotide sequence encoding all or part of CSF1 to a polynucleotide sequence encoding all or part of granzyme B, and translation of the joined polynucleotide sequence. The fusion protein has functional properties derived from CSF1 and granzyme B.

The fusion protein may be of any size. That is, the fusion protein may be any number of amino acids in length. For example, the fusion protein may be about 50 to about 1000 amino acids in length, such as about 100 to about 900, about 150 to about 850, about 200 to about 800, about 250 to about 750, about 300 to about 700, about 350 to about 650, about 400 to about 600, about 450 to about 550, or about 500 amino acids in length. Preferably, the fusion protein may be from about 350 to about 500 amino acids in length, such as about 360 to about 480, about 370 to about 470, about 380 to about 460, about 390 to about 450, about 400 to about 440, about 410 to about 430, or about 420 amino acids in length. The fusion protein may, for example, be about 390 to about 470 amino acids in length, such as about 390 to about 440 amino acids in length. The fusion protein may, for example, be about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 amino acids in length.

The term “protein” includes not only molecules in which amino acid residues are joined by peptide (—CO—NH—) linkages but also molecules in which the peptide bond is reversed. Such retro-inverso peptidomimetics, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH2 may be modified to —NH (Me) or —N(Me)₂).

The term “protein” also includes protein variants that increase or decrease the half-life of the protein in vivo. Examples of analogues capable of increasing the half-life of proteins described herein include peptoid analogues of the proteins, D-amino acid derivatives of the proteins, and peptide-peptoid hybrids. A further aspect described herein comprises D-amino acid forms of the protein. The preparation of proteins using D-amino acids rather than L-amino acids greatly decreases any unwanted breakdown of such an agent by normal metabolic processes, decreasing the amounts of agent which needs to be administered, along with the frequency of its administration.

The fusion protein of the present disclosure comprises a CSF1 portion and a granzyme B portion. The fusion protein may, for example, consist of a CSF1 portion and a granzyme B portion. The fusion protein may, for example, comprise components additional to the CSF1 portion and granzyme B portion. For example, the fusion protein may further comprise (i) an epitope tag, (ii) a fusogenic peptide, and/or (iii) a leader sequence. For instance, the fusion protein may further comprise (i); (ii); (iii); (i) and (ii); (i) and (iii); (ii) and (iii); or (i), (ii) and (iii). CSF1 portions, granzyme B portions, epitope tags, fusogenic peptides and leader sequences are described in detail below. Any of the components of the fusion protein may be joined by a linker, such as a cleavable linker. Linkers are also described in detail below.

### CSF1 Portion

Colony stimulating factor 1 (CSF1; otherwise known as macrophage colony-stimulating factor (M-CSF)) is a secreted cytokine that binds to colony stimulating factor 1 receptor (CSF1R; otherwise known as macrophage colony-stimulating factor receptor (M-CSFR) or CD115). CSF1 is a hematopoietic growth factor that is involved in the proliferation, differentiation, and survival of monocytes, macrophages, and bone marrow progenitor cells. CSF1 stimulates phagocytic, chemotactic activity, and tumour cell cytotoxicity in macrophages and monocytes. CSF1 also has functions outside of the monocyte/macrophage cell lineage, such as in immunology, metabolism, fertility and pregnancy.

The fusion protein comprises a colony stimulating factor 1 (CSF1) portion. The CSF1 portion is a CSF1 protein, or a variant, fragment, or variant fragment thereof that specifically binds to CSF1 receptor. The CSF1 protein may be a CSF1 protein from any species. Preferably, the CSF1 protein is human CSF1 protein.

The CSF1 protein may, for example, comprise or consist of the amino acid sequence of SEQ ID NO: 1. SEQ ID NO: 1 is the amino acid sequence of wild-type human CSF1. The term “wild-type” refers to a gene or gene product isolated from a naturally occurring source. A wild-type gene or gene product is that which is most frequently observed in a population and is thus arbitrarily designed the “normal” or “wild-type” form of the gene or gene product. The CSF1 portion may comprise SEQ ID NO: 1. The CSF1 portion may comprise a variant of SEQ ID NO: 1. The CSF1 portion may comprise a fragment of SEQ ID NO: 1. The CSF1 portion may comprise a variant fragment of SEQ ID NO: 1. The CSF1 portion may consist of SEQ ID NO: 1. The CSF1 portion may consist of a variant of SEQ ID NO: 1. The CSF1 portion may consist of a fragment of SEQ ID NO: 1. The CSF1 portion may consist of a variant fragment of SEQ ID NO: 1.

The CSF1 protein may, for example, comprise or consist of the amino acid sequence of SEQ ID NO: 2. SEQ ID NO: 2 is a fragment of wild-type human CSF1 that consists of amino acids 33 to 181 of wild-type human CSF1. That is, SEQ ID NO: 2 consists of amino acids 33 to 181 of SEQ ID NO: 1. The CSF1 portion may comprise SEQ ID NO: 2. The CSF1 portion may comprise a variant of SEQ ID NO: 2. The CSF1 portion may comprise a fragment of SEQ ID NO: 2. The CSF1 portion may comprise a variant fragment of SEQ ID NO: 2. The CSF1 portion may consist of SEQ ID NO: 2. The CSF1 portion may consist of a variant of SEQ ID NO: 2. The CSF1 portion may consist of a fragment of SEQ ID NO: 2. The CSF1 portion may consist of a variant fragment of SEQ ID NO: 2.

The CSF1 protein may, for example, comprise or consist of the amino acid sequence of SEQ ID NO: 3. SEQ ID NO: 3 is a fragment of wild-type human CSF1 that consists of amino acids 33 to 196 of wild-type human CSF1. That is, SEQ ID NO: 3 consists of amino acids 33 to 196 of SEQ ID NO: 1. The CSF1 portion may comprise SEQ ID NO: 3. The CSF1 portion may comprise a variant of SEQ ID NO: 3. The CSF1 portion may comprise a fragment of SEQ ID NO: 3. The CSF1 portion may comprise a variant fragment of SEQ ID NO: 3. The CSF1 portion may consist of SEQ ID NO: 3. The CSF1 portion may consist of a variant of SEQ ID NO: 3. The CSF1 portion may consist of a fragment of SEQ ID NO: 3. The CSF1 portion may consist of a variant fragment of SEQ ID NO: 3. SEQ ID NO: 3 (relating to amino acids 33 to 196 of wild-type human CSF1 (SEQ ID NO: 1)) is extended relative to SEQ ID NO: 2 (relating to amino acids 33 to 181 of wild-type human CSF1 (SEQ ID NO: 1)). Inclusion of amino acids 182 to 196 of wild-type human CSF1 in SEQ ID NO: 3 promotes release of the fusion protein from endosomal vesicles.

The CSF1 protein may, for example, comprise or consist of the amino acid sequence of SEQ ID NO: 4. SEQ ID NO: 4 is a C63S/M59R variant of a fragment of wild-type human CSF1 that consists of amino acids 33 to 181 of wild-type human CSF1. That is, SEQ ID NO: 4 is a C63S/M59R variant of SEQ ID NO: 2. In other words, SEQ ID NO: 4 represents SEQ ID NO: 2 with the substitutions C63S and M59R. Positions “63” and “59” relate to the relevant position in wild-type human CSF1. The CSF1 portion may comprise SEQ ID NO: 4. The CSF1 portion may comprise a variant of SEQ ID NO: 4. The CSF1 portion may comprise a fragment of SEQ ID NO: 4. The CSF1 portion may comprise a variant fragment of SEQ ID NO: 4. The CSF1 portion may consist of SEQ ID NO: 4. The CSF1 portion may consist of a variant of SEQ ID NO: 4. The CSF1 portion may consist of a fragment of SEQ ID NO: 4. The CSF1 portion may consist of a variant fragment of SEQ ID NO: 4. Introduction of the substitutions C63S and M59R inhibits dimerization of the fusion protein, which may be beneficial.

The CSF1 protein may, for example, comprise or consist of the amino acid sequence of SEQ ID NO: 5. SEQ ID NO: 5 is a C63S/M59R variant of a fragment of wild-type human CSF1 that consists of amino acids 33 to 196 of wild-type human CSF1. That is, SEQ ID NO: 5 is a C63S/M59R variant of SEQ ID NO: 3. In other words, SEQ ID NO: 5 represents SEQ ID NO: 3 with the substitutions C63S and M59R. Positions “63” and “59” relate to the relevant position in wild-type human CSF1. The CSF1 portion may comprise SEQ ID NO: 5. The CSF1 portion may comprise a variant of SEQ ID NO: 5. The CSF1 portion may comprise a fragment of SEQ ID NO: 5. The CSF1 portion may comprise a variant fragment of SEQ ID NO: 5. The CSF1 portion may consist of SEQ ID NO: 5. The CSF1 portion may consist of a variant of SEQ ID NO: 5. The CSF1 portion may consist of a fragment of SEQ ID NO: 5. The CSF1 portion may consist of a variant fragment of SEQ ID NO: 5. As set out above, introduction of the substitutions C63S and M59R inhibits dimerization of the fusion protein, which may be beneficial.

A variant of a CSF1 protein (such as a variant of SEQ ID NO: 1, 2, 3, 4 or 5) encompasses peptides, oligopeptides, polypeptides, proteins and enzymes that (a) have at least one amino acid substitution, at least one amino acid deletion and/or at least one amino acid insertion relative to the CSF1 protein, and (b) are capable of specifically binding to CSF1 receptor. The variant may, for example, comprise (i) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid substitutions relative to the CSF1 protein. The variant may, for example, comprise (ii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid deletions relative to the CSF1 protein. The variant may, for example, comprise (iii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid insertions relative to the CSF1 protein. The variant may comprise (i); (ii); (iii); (i) and (ii); (i) and (iii); (ii) and (iii); or (i), (ii) and (iii). The variant may, for example, have at least 50%, at least 60%, at least 65%, at least 70%, at least 75%, at least 80%, at least 85%, at least 90%, at least 95%, at least 97%, at least 98%, or at least 99% sequence identity to the amino acid sequence of the corresponding wild-type protein. Determination of percentage identity is described in detail above.

Amino acid substitutions comprised in the variant may be conservative substitutions. Conservative substitutions replace an amino acid with another amino acid of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, |
| | | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Methods for substituting, introducing or deleting naturally-occurring amino acids are well known in the art. For instance, methionine (M) may be substituted with arginine (R) by replacing the codon for methionine (ATG) with a codon for arginine (CGT) at the relevant position in a polynucleotide encoding the variant. Methionine may be introduced by inserting the codon for methionine (ATG) in-frame at the relevant position in a polynucleotide encoding the variant. Methionine may be deleted by deleting the codon for methionine (ATG) at the relevant position in a polynucleotide encoding the variant.

A fragment of a CSF1 protein (such as a fragment of SEQ ID NO: 1, 2, 3, 4 or 5) encompasses peptides, oligopeptides, polypeptides, proteins and enzymes that (a) comprise or consist of an amino acid sequence that is derived by truncation at the N-terminus and/or C-terminus of the sequence of the CSF1 protein, and (b) are capable of specifically binding to CSF1 receptor. The fragment may, for example, comprise or consist of about 1% to about 99% of the sequence of the CSF1 protein. For instance, the fragment comprise or consist of about 2% to about 98%, about 3% to about 97%, about 4% to about 96%, about 5% to about 95%, about 6% to about 94%, about 7% to about 93%, about 8% to about 92%, about 9% to about 91%, about 10% to about 90%, about 15% to about 85%, about 20% to about 80%, about 25% to about 75%, about 30% to about 70%, about 35% to about 65%, about 40% to about 60%, about 45% to about 55% of the sequence of the CSF1 protein. For example, the fragment comprise or consist of about 10%, about 20%, about 30%, about 40%, about 50%, about 60%, about 70%, about 80%, or about 90% of the sequence of the CSF1 protein. The fragment may be of any size. That is, the fragment may be any number of amino acids in length. For example, the fragment may be about 20 to about 200 amino acids in length, such as about 30 to about 190, about 40 to about 180, about 50 to about 160, about 60 to about 150, about 70 to about 140, about 80 to about 130, about 90 to about 120, or about 100 to about 110 amino acids in length. For instance, the fragment may be about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 amino acids in length.

A variant fragment of a CSF1 protein (such as a variant fragment of SEQ ID NO: 1, 2, 3, 4 or 5) encompasses peptides, oligopeptides, polypeptides, proteins and enzymes that (a) have at least one amino acid substitution, at least one amino acid deletion and/or at least one amino acid insertion relative to a fragment of the CSF1 protein, and (b) are capable of specifically binding to CSF1 receptor. The fragment may be any fragment of a CSF1 protein defined above. The variant may, for example, comprise (i) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid substitutions relative to the fragment. The variant may, for example, comprise (ii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid deletions relative to the fragment. The variant may, for example, comprise (iii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid insertions relative to the fragment. The variant may comprise (i); (ii); (iii); (i) and (ii); (i) and (iii); (ii) and (iii); or (i), (ii) and (iii). The variant may, for example, have at least 50%, at least 60%, at least 65%, at least 70%, at least 75%, at least 80%, at least 85%, at least 90%, at least 95%, at least 97%, at least 98%, or at least 99% sequence identity to the amino acid sequence of the fragment. Determination of percentage identity is described in detail above. Amino acid substitutions comprised in the variant fragment may be conservative substitutions. Conservative substitutions are described in detail above.

### Granzyme B Portion

Granzyme B is a serine protease commonly found in the granules of cytotoxic T cells and natural killer cells (NK cells). It is secreted to mediate apoptosis in target cells.

The fusion protein comprises a granzyme B portion. The granzyme B portion is a granzyme B protein, or a variant, fragment, or variant fragment thereof that has serine protease activity. The granzyme B protein may be a granzyme B protein from any species. Preferably, the granzyme B protein is human granzyme B protein.

The granzyme B protein may, for example, comprise or consist of the amino acid sequence of SEQ ID NO: 6. SEQ ID NO: 6 is the amino acid sequence of wild-type human granzyme B. The term “wild-type” is defined above.

The granzyme B portion may comprise SEQ ID NO: 6. The granzyme B portion may comprise a variant of SEQ ID NO: 6. The granzyme B portion may comprise a fragment of SEQ ID NO: 6. The granzyme B portion may comprise a variant fragment of SEQ ID NO: 6. The granzyme B portion may consist of SEQ ID NO: 6. The granzyme B portion may consist of a variant of SEQ ID NO: 6. The granzyme B portion may consist of a fragment of SEQ ID NO: 6. The granzyme B portion may consist of a variant fragment of SEQ ID NO: 6.

The granzyme B protein may, for example, comprise or consist of the amino acid sequence of SEQ ID NO: 7. SEQ ID NO: 7 is a fragment of wild-type human granzyme B that consists of amino acids 21 to 247 of wild-type human granzyme B. That is, SEQ ID NO: 7 consists of amino acids 21 to 247 of SEQ ID NO: 6. The granzyme B portion may comprise SEQ ID NO: 7. The granzyme B portion may comprise a variant of SEQ ID NO: 7. The granzyme B portion may comprise a fragment of SEQ ID NO: 7. The granzyme B portion may comprise a variant fragment of SEQ ID NO: 7. The granzyme B portion may consist of SEQ ID NO: 7. The granzyme B portion may consist of a variant of SEQ ID NO: 7. The granzyme B portion may consist of a fragment of SEQ ID NO: 7. The granzyme B portion may consist of a variant fragment of SEQ ID NO: 7.

The granzyme B protein may, for example, comprise or consist of the amino acid sequence of SEQ ID NO: 8. SEQ ID NO: 8 is a R221K variant of a fragment of wild-type human granzyme B that consists of amino acids 21 to 247 of wild-type human granzyme B. That is, SEQ ID NO: 8 is a R221K variant of SEQ ID NO: 7. In other words, SEQ ID NO: 8 represents SEQ ID NO: 7 with the substitution R221K. The granzyme B portion may comprise SEQ ID NO: 8. The granzyme B portion may comprise a variant of SEQ ID NO: 8. The granzyme B portion may comprise a fragment of SEQ ID NO: 8. The granzyme B portion may comprise a variant fragment of SEQ ID NO: 8. The granzyme B portion may consist of SEQ ID NO: 8. The granzyme B portion may consist of a variant of SEQ ID NO: 8. The granzyme B portion may consist of a fragment of SEQ ID NO: 8. The granzyme B portion may consist of a variant fragment of SEQ ID NO: 8. Introduction of the R221K substitution confers resistance to serpin B9, a natural inhibitor of granzyme B.

The granzyme B protein may, for example, comprise or consist of the amino acid sequence of SEQ ID NO: 9. SEQ ID NO: 9 is a R116A/R120A/R122A/K241A/K242A/K245A/R246A variant of a fragment of wild-type human granzyme B that consists of amino acids 21 to 247 of wild-type human granzyme B. That is, SEQ ID NO: 9 is a R116A/R120A/R122A/K241A/K242A/K245A/R246A variant of SEQ ID NO: 7. In other words, SEQ ID NO: 9 represents SEQ ID NO: 7 with the substitutions R116A, R120A, R122A, K241A, K242A, K245A and R246A. The granzyme B portion may comprise SEQ ID NO: 9. The granzyme B portion may comprise a variant of SEQ ID NO: 9. The granzyme B portion may comprise a fragment of SEQ ID NO: 9. The granzyme B portion may comprise a variant fragment of SEQ ID NO: 9. The granzyme B portion may consist of SEQ ID NO: 9. The granzyme B portion may consist of a variant of SEQ ID NO: 9. The granzyme B portion may consist of a fragment of SEQ ID NO: 9. The granzyme B portion may consist of a variant fragment of SEQ ID NO: 9. Introduction of the substitutions R116A, R120A, R122A, K241A, K242A, K245A and R246A substitutions minimises the positive potential of granzyme B on the cell surface, to reduce non-specific binding and thus off-target effects.

A variant of a granzyme B protein (such as a variant of SEQ ID NO: 6, 7, 8 or 9) encompasses peptides, oligopeptides, polypeptides, proteins and enzymes that (a) have at least one amino acid substitution, at least one amino acid deletion and/or at least one amino acid insertion relative to the granzyme B protein, and (b) have serine protease activity. The variant may, for example, comprise (i) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid substitutions relative to the granzyme B protein. The variant may, for example, comprise (ii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid deletions relative to the granzyme B protein. The variant may, for example, comprise (iii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid insertions relative to the granzyme B protein. The variant may comprise (i); (ii); (iii); (i) and (ii); (i) and (iii); (ii) and (iii); or (i), (ii) and (iii). The variant may, for example, have at least 50%, at least 60%, at least 65%, at least 70%, at least 75%, at least 80%, at least 85%, at least 90%, at least 95%, at least 97%, at least 98%, or at least 99% sequence identity to the amino acid sequence of the corresponding wild-type protein. Determination of percentage identity is described in detail above. Amino acid substitutions comprised in the variant may be conservative substitutions. Conservative substitutions are described in detail above.

A fragment of a granzyme B protein (such as a fragment of SEQ ID NO: 1 SEQ ID NO: 6, 7, 8 or 9) encompasses peptides, oligopeptides, polypeptides, proteins and enzymes that (a) comprise or consist of an amino acid sequence that is derived by truncation at the N-terminus and/or C-terminus of the sequence of the granzyme B protein, and (b) have serine protease activity. The fragment may, for example, comprise or consist of about 1% to about 99% of the sequence of the granzyme B protein. For instance, the fragment comprise or consist of about 2% to about 98%, about 3% to about 97%, about 4% to about 96%, about 5% to about 95%, about 6% to about 94%, about 7% to about 93%, about 8% to about 92%, about 9% to about 91%, about 10% to about 90%, about 15% to about 85%, about 20% to about 80%, about 25% to about 75%, about 30% to about 70%, about 35% to about 65%, about 40% to about 60%, about 45% to about 55% of the sequence of the granzyme B protein. For example, the fragment comprise or consist of about 10%, about 20%, about 30%, about 40%, about 50%, about 60%, about 70%, about 80%, or about 90% of the sequence of the granzyme B protein. The fragment may be of any size. That is, the fragment may be any number of amino acids in length. For example, the fragment may be about 20 to about 200 amino acids in length, such as about 30 to about 190, about 40 to about 180, about 50 to about 160, about 60 to about 150, about 70 to about 140, about 80 to about 130, about 90 to about 120, or about 100 to about 110 amino acids in length. For instance, the fragment may be about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 amino acids in length.

A variant fragment of a granzyme B protein (such as a variant fragment of SEQ ID NO: SEQ ID NO: 6, 7, 8 or 9) encompasses peptides, oligopeptides, polypeptides, proteins and enzymes that (a) have at least one amino acid substitution, at least one amino acid deletion and/or at least one amino acid insertion relative to a fragment of the granzyme B protein, and (b) have serine protease activity. The fragment may be any fragment of a granzyme B protein defined above. The variant may, for example, comprise (i) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid substitutions relative to the fragment. The variant may, for example, comprise (ii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid deletions relative to the fragment. The variant may, for example, comprise (iii) one or more (such as two or more, three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 75 or more, or 100 or more) amino acid insertions relative to the fragment. The variant may comprise (i); (ii); (iii); (i) and (ii); (i) and (iii); (ii) and (iii); or (i), (ii) and (iii). The variant may, for example, have at least 50%, at least 60%, at least 65%, at least 70%, at least 75%, at least 80%, at least 85%, at least 90%, at least 95%, at least 97%, at least 98%, or at least 99% sequence identity to the amino acid sequence of the fragment. Determination of percentage identity is described in detail above. Amino acid substitutions comprised in the variant fragment may be conservative substitutions. Conservative substitutions are described in detail above.

### Epitope Tag

The fusion protein may comprise an epitope tag. An epitope tag is a known epitope that may be engineered onto a protein, for instance at the C-terminus or the N-terminus. An epitope is the part of a peptide antigen that is recognised by one or more antigen-binding proteins, such as an antibody, a B cell receptor or a T cell receptor. An epitope tag may, for example, comprise an epitope for a commercially-available antibody. The antibody may, for example, be used detect the epitope tag (and thus the protein onto which the epitope tag is engineered). For instance, an epitope tag may be useful in immunoassays, such as western blotting, immunofluorescence, immunoprecipitation, immunohistochemistry, and immunocytochemistry. The antibody may, for example be used to purify the protein onto which the epitope tag is engineered, for instance by affinity purification. Thus, an epitope tag may be useful in isolating a tagged protein.

The epitope tag may be cleavably joined to the fusion protein. Therefore, the epitope tag may, for instance, be used to detect or isolate the fusion protein and then removed. In the event that the fusion protein is produced in vitro for subsequent administration to an individual, the epitope tag is typically removed prior to administration. Similarly, the epitope tag may be excluded from the fusion protein when it is intended to administer an individual with a virus or T cell comprising a polynucleotide encoding the fusion protein.

The epitope tag may be cleavably joined to the fusion protein in any suitable manner. For example, the epitope tag may comprise or consist of a cleavage site targeted by a cleavage enzyme. The epitope tag may, for example, comprise or consist of an enterokinase cleavage site. The enterokinase cleavage site may, for example, comprise or consist of the amino acid sequence DYKDDDDK (SEQ ID NO: 10). As set out below SEQ ID NO: 10 corresponds to a FLAG-tag that may be comprised in the fusion protein. A FLAG-tag is a type of epitope tag.

The epitope tag is typically about 5 to about 25 amino acids in length. For instance, the epitope tag may be about 6 to about 24, about 7 to about 23, about 8 to about 22, about 9 to about 21, about 10 to about 20, about 11 to about 19, about 12 to about 18, about 13 to about 17, about 14 to about 16, or about 15 amino acids in length. The epitope tag may, for instance, be about 5, 6, 7, 8, 9, 10, 11, 12, 13, 14, 15, 16, 17, 18, 19, 20, 21, 22, 23, 24 or 25 amino acids in length. Preferably, the epitope tag is about 6 to about 10 amino acids in length, such as about 7 to about amino acids in length to about 8 amino acids in length.

The epitope tag may, for example, comprise a FLAG-tag. The epitope tag may consist of a FLAG-tag. The FLAG-tag may comprise SEQ ID NO: 10. The FLAG-tag may consist of SEQ ID NO: 10. As set out above, SEQ ID NO: 10 (DYKDDDDK) provides a cleavage site for an enterokinase.

The FLAG-tag is well-known in the art, and has several benefits as an epitope tag. For example, the FLAG-tag's hydrophilic nature renders it less likely to denature or inactivate tagged proteins than other common epitope tags. Furthermore, FLAG-tags can be removed readily from proteins once no longer required (e.g. post-isolation) by treatment with the specific protease, enterokinase.

The epitope tag may be incorporated to the fusion protein at any location. For example, the epitope tag may be located at or near the C-terminus of the fusion protein. Preferably, the epitope tag is located at or near the N-terminus of the fusion protein. Accordingly, in a polynucleotide (e.g. mRNA) encoding the fusion protein, the nucleic acid sequence encoding the epitope tag may be located at or near the 3′ end of the polynucleotide. Preferably, the nucleic acid sequence encoding the epitope tag may be located at or near the 5′ end of the polynucleotide. For example, the nucleic acid sequence encoding the epitope tag may be located downstream (i.e. 3′) of a Kozak consensus sequence. The nucleic acid sequence encoding the epitope tag may be located downstream of a leader sequence. The nucleic acid sequence encoding the epitope tag may be located downstream of a Kozak consensus sequence and a leader sequence.

### Fusogenic Peptide

The fusion protein may comprise a fusogenic peptide. Inclusion of a fusogenic peptide in the fusion protein promotes release of the fusion protein from endosomal vesicles.

A fusogenic peptide is a peptide that promotes membrane fusion. In the context of the present disclosure, “membrane fusion” refers to the fusion of biological membranes (or phospholipid bilayers) each defining a cell, organelle, or extracellular vesicle. The organelle may, for example, be a component of the endomembrane system, such as the nuclear membrane, the endoplasmic reticulum, the Golgi apparatus, a lysosome or an endosomes.

The fusogenic peptide is typically about 15 to about 35 amino acids in length. For instance, the fusogenic peptide may be about 20 to about 30, or about 25 amino acids in length. The fusogenic peptide may, for instance, be about 15, 16, 16, 18, 19, 20, 21, 22, 23, 24, 25, 26, 27, 28, 29, 30, 31, 32, 33, 34 or 35 amino acids in length.

The fusogenic peptide may, for example, form an alpha helix motif. The alpha-helix motif has been reported to confer fusogenic ability. An exemplary fusogenic peptide that forms an alpha helix motif is SEQ ID NO: 11. The fusogenic peptide may comprise SEQ ID NO: 11. The fusogenic peptide may consist of SEQ ID NO: 11. However, other fusogenic peptides are known in the art and are expected to likewise promote release of the fusion protein from endosomal vesicles. Therefore, any fusogenic peptide may be included in the fusion protein.

The fusogenic peptide may be incorporated to the fusion protein at any location. The fusogenic peptide may, for example, be located at or near the N-terminus of the fusion protein, or at or near the C-terminus of the fusion protein. The fusogenic peptide may, for example, be joined to the CSF1 portion or to the granzyme B portion. Preferably, the fusogenic peptide is joined to the CSF1 portion. The CSF1 portion is typically found towards the C-terminus of the fusion protein.

Accordingly, in a polynucleotide (e.g. mRNA) encoding the fusion protein, the nucleic acid sequence encoding the fusogenic peptide may be located at any point within the polynucleotide. For example, the nucleic acid sequence encoding the fusogenic peptide may be located the at or near the 5′ end of the polynucleotide, or at or near the 3′ end of the polynucleotide. For example, the nucleic acid sequence encoding the fusogenic peptide may be located downstream (i.e. 3′) of a nucleic acid sequence encoding the CSF1 portion. The nucleic acid sequence encoding the fusogenic peptide may be located downstream of a nucleic acid sequence encoding the granzyme B portion. The nucleic acid sequence encoding the fusogenic peptide may be located downstream of a nucleic acid sequence encoding the granzyme B portion and the CSF1 portion. The nucleic acid sequence encoding the fusogenic peptide may be located upstream (i.e. 5′) of the 3′ untranslated region. The nucleic acid sequence encoding the fusogenic peptide may be located upstream of a polyA tail.

### Leader Sequence

The fusion protein may comprise a leader sequence. A leader sequence is a peptide that directs translocation of a protein in which it is comprised. For instance, a leader sequence may direct a protein through the endoplasmic reticulum (ER)-Golgi processing pathway, and onwards to the cell surface for secretion. A leader sequence may alternatively be known as a leader peptide, signal sequence, signal peptide, targeting signal, localisation signal, localisation sequence or transit peptide, for example.

The leader sequence may be cleavably joined to the fusion protein. Therefore, the leader sequence may, for instance, be used to direct the fusion protein through the secretory pathway then removed. In the event that the fusion protein is produced in vitro for subsequent administration to an individual, the leader sequence is typically removed prior to administration. Similarly, the leader sequence may be excluded from the fusion protein when it is intended to administer an individual with a virus or T cell comprising a polynucleotide encoding the fusion protein.

The leader sequence may be cleavably joined to the fusion protein by any suitable means, such as a cleavable linker. In one exemplary aspect described herein, the leader sequence may be cleavably joined to the fusion protein by a cleavable epitope tag. The cleavable epitope tag may comprise or consist of a cleavage site targeted by a cleavage enzyme. The epitope tag may, for example, comprise or consist of an enterokinase cleavage site. The enterokinase cleavage site may, for example, comprise or consist of the amino acid sequence DYKDDDDK (SEQ ID NO: 10). As set out below SEQ ID NO: 10 corresponds to a FLAG-tag that may be comprised in the fusion protein. The cleavable epitope tag may therefore comprise or consist of a FLAG-tag, such as a FLAG-tag that comprises or consists of SEQ ID NO: 10.

The leader sequence is typically about 10 to about 30 amino acids in length. For instance, the leader sequence may be about 15 to about 25, or about 20 amino acids in length. The leader sequence may, for instance, be about 10, 11, 12, 13, 14, 15, 16, 17, 18, 19, 20, 21, 22, 23, 24, 25, 26, 27, 28, 29 or 30 amino acids in length.

An exemplary leader sequence is represented by SEQ ID NO: 12. The leader sequence may comprise SEQ ID NO: 12. The leader sequence may consist of SEQ ID NO: 12. However, other leader sequences are well-known in the art and are expected to direct translocation in the same way as SEQ ID NO: 12. Therefore, any leader sequence may be included in the fusion protein.

The leader sequence may be incorporated to the fusion protein at any location. The leader sequence may, for example, be located at the N-terminus of the fusion protein, or at or the C-terminus of the fusion protein. Typically, the leader sequence is located at the N-terminus of the fusion protein.

Accordingly, in a polynucleotide (e.g. mRNA) encoding the fusion protein, the nucleic acid sequence encoding the leader sequence may be located at any point within the polynucleotide. For example, the nucleic acid sequence encoding the leader sequence may be located the at or near the 5′ end of the polynucleotide, or at or near the 3′ end of the polynucleotide. For example, the nucleic acid sequence encoding the leader sequence may be located downstream (i.e. 3′) of a Kozak consensus sequence. The nucleic acid sequence encoding the leader sequence may be located upstream (i.e. 5′) of a nucleic acid sequence encoding the granzyme B portion. The nucleic acid sequence encoding the leader sequence may be located upstream of a nucleic acid sequence encoding the CSF1 portion.

The nucleic acid sequence encoding the leader sequence may be located upstream of a nucleic acid sequence encoding the granzyme B portion CSF1 portion.

### Linker

The fusion protein may comprise one or more linkers. For example, the fusion protein may comprise one, two, three or four linkers. Each of the linkers joins two components of the fusion protein. As set out above, the components of the fusion protein include at least (a) a CSF1 portion and (b) a granzyme B portion. The fusion protein may comprise one or more further components selected from (c) an epitope tag, (d) a fusogenic peptide, and (e) a leader sequence.

If a linker is present in the fusion protein, it typically joins the CSF1 portion to the granzymes B portion. However, any two components of the fusion protein may in principle be joined by a linker. For instance, the linker may join (a) and (b), (a) and (c), (a) and (d), (a) and (e), (b) and (c), (b) and (d), (b) and (e), (c) and (d), (c) and (e), or (d) and (e). In practice, the components joinable by a linker will depend on the order of the components within the fusion protein. The joined components are joined along peptide backbone of the fusion protein.

The linker is a peptide linker. That is, the linker comprises a peptide. Typically, the linker is a peptide that is about 15 to about 25 amino acids in length. For instance, the linker may be a peptide that is about 16 to about 24, about 17 to about 23, about 18 to about 22, about 19 to about 21, or about 20 amino acids in length. For example, the linker may be about 15, 16, 17, 18, 19, 20, 21, 22, 23, 24 or 25 amino acids in length. Suitable peptide linkers are known in the art.

The linker may be a flexible linker. Suitable flexible linkers are known in the art, and include linkers that comprise at least one glycine residue and at least one serine residue. For instance, a linker may comprise a repeated sequence of glycine and serine residues. In one aspect, the linker comprises the sequence GGGGS (SEQ ID NO: 13), or repeats of the sequence GGGGS such as (GGGGS)₄. In one aspect, the linker comprises the sequence GGGS (SEQ ID NO: 35), or repeats of the sequence GGGS. Use of a flexible linker may allow a certain degree of movement or interaction between domains joined by the linker. This is conferred by the flexibility of the linker itself. In essence, a flexible linker may allow for mobility of the connected functional domains.

The linker may be a cleavable linker. A cleavable linker is linker that is cleavable by an enzyme, such as a protease. Suitable cleavable linkers are known in the art. One exemplary cleavable linker is represented by SEQ ID NO: 33. A cleavable linker may comprise SEQ ID NO: 33. For example, an exemplary cleavable linker is represented by SEQ ID NO: 14. Another exemplary cleavable linker is represented by SEQ ID NO: 34. Use of a cleavable linker may (a) reduce steric hindrance, (b) improve bioactivity, and/or (c) allow for independent actions and/or metabolism of individual domains, following linker cleavage.

### Exemplary Fusion Proteins

The fusion protein may comprise (for instance, from N-terminal to C-terminal):
I. A granzyme B portion, a flexible linker, and a CSF1 portion. The flexible linker may be (GGGGS)₄.
II. An epitope tag, a granzyme B portion, a flexible linker, and a CSF1 portion. The epitope tag may be a FLAG-tag of SEQ ID NO: 10. The flexible linker may be (GGGGS)₄. The epitope tag may be a FLAG-tag of SEQ ID NO: 10 and the flexible linker may be (GGGGS)₄.
III. A granzyme B portion, a flexible linker, a CSF1 portion, and a fusogenic peptide. The flexible linker may be (GGGGS)₄. The fusogenic peptide may be a fusogenic peptide of SEQ ID NO: 11. The flexible linker may be (GGGGS)₄, and the fusogenic peptide may be a fusogenic peptide of SEQ ID NO: 11.
IV. An epitope tag, a granzyme B portion, a flexible linker, a CSF1 portion, and a fusogenic peptide. The epitope tag may be a FLAG-tag of SEQ ID NO: 10. The flexible linker may be (GGGGS)₄. The fusogenic peptide may be a fusogenic peptide of SEQ ID NO: 11. The flexible linker may be (GGGGS)₄. The epitope tag may be a FLAG-tag of SEQ ID NO: 10, the flexible linker may be (GGGGS)₄, and the fusogenic peptide may be a fusogenic peptide of SEQ ID NO: 11.
V. A granzyme B portion, a cleavable linker, and a CSF1 portion. The cleavable linker may comprise or consist of SEQ ID NO: 14. The cleavable linker may comprise or consist of SEQ ID NO: 33 or 34.
VI. An epitope tag, a granzyme B portion, a cleavable linker, and a CSF1 portion. The epitope tag may be a FLAG-tag of SEQ ID NO: 10. The cleavable linker may comprise or consist of SEQ ID NO: 14. The epitope tag may be a FLAG-tag of SEQ ID NO: 10 and the cleavable linker may comprise or consist of SEQ ID NO: 14. The cleavable linker may comprise or consist of SEQ ID NO: 33 or 34.

In any of I to VI above, the CSF1 portion may, for example, comprise SEQ ID NO: 2, 3, 4 or 5. The granzyme B portion may, for example, comprise SEQ ID NO: 6, 7, 8 or 9. The CSF1 portion may comprise SEQ ID NO: 2 and the granzyme B portion may comprise SEQ ID NO: 6. The CSF1 portion may comprise SEQ ID NO: 2 and the granzyme B portion may comprise SEQ ID NO: 7. The CSF1 portion may comprise SEQ ID NO: 2 and the granzyme B portion may comprise SEQ ID NO: 8. The CSF1 portion may comprise SEQ ID NO: 2 and the granzyme B portion may comprise SEQ ID NO: 9. The CSF1 portion may comprise SEQ ID NO: 3 and the granzyme B portion may comprise SEQ ID NO: 6. The CSF1 portion may comprise SEQ ID NO: 3 and the granzyme B portion may comprise SEQ ID NO: 7. The CSF1 portion may comprise SEQ ID NO: 3 and the granzyme B portion may comprise SEQ ID NO: 8. The CSF1 portion may comprise SEQ ID NO: 3 and the granzyme B portion may comprise SEQ ID NO: 9. The CSF1 portion may comprise SEQ ID NO: 4 and the granzyme B portion may comprise SEQ ID NO: 6. The CSF1 portion may comprise SEQ ID NO: 4 and the granzyme B portion may comprise SEQ ID NO: 7. The CSF1 portion may comprise SEQ ID NO: 4 and the granzyme B portion may comprise SEQ ID NO: 8. The CSF1 portion may comprise SEQ ID NO: 4 and the granzyme B portion may comprise SEQ ID NO: 9. The CSF1 portion may comprise SEQ ID NO: 5 and the granzyme B portion may comprise SEQ ID NO: 6. The CSF1 portion may comprise SEQ ID NO: 5 and the granzyme B portion may comprise SEQ ID NO: 7. The CSF1 portion may comprise SEQ ID NO: 5 and the granzyme B portion may comprise SEQ ID NO: 8. The CSF1 portion may comprise SEQ ID NO: 5 and the granzyme B portion may comprise SEQ ID NO: 9.

In any of I to VI above, the CSF1 portion may, for example, consist of SEQ ID NO: 2, 3, 4 or 5. The granzyme B portion may, for example, consist of SEQ ID NO: 6, 7, 8 or 9. The CSF1 portion may consist of SEQ ID NO: 2 and the granzyme B portion may consist of SEQ ID NO: 6. The CSF1 portion may consist of SEQ ID NO: 2 and the granzyme B portion may consist of SEQ ID NO: 7. The CSF1 portion may consist of SEQ ID NO: 2 and the granzyme B portion may consist of SEQ ID NO: 8. The CSF1 portion may consist of SEQ ID NO: 2 and the granzyme B portion may consist of SEQ ID NO: 9. The CSF1 portion may consist of SEQ ID NO: 3 and the granzyme B portion may consist of SEQ ID NO: 6. The CSF1 portion may consist of SEQ ID NO: 3 and the granzyme B portion may consist of SEQ ID NO: 7. The CSF1 portion may consist of SEQ ID NO: 3 and the granzyme B portion may consist of SEQ ID NO: 8. The CSF1 portion may consist of SEQ ID NO: 3 and the granzyme B portion may consist of SEQ ID NO: 9. The CSF1 portion may consist of SEQ ID NO: 4 and the granzyme B portion may consist of SEQ ID NO: 6. The CSF1 portion may consist of SEQ ID NO: 4 and the granzyme B portion may consist of SEQ ID NO: 7. The CSF1 portion may consist of SEQ ID NO: 4 and the granzyme B portion may consist of SEQ ID NO: 8. The CSF1 portion may consist of SEQ ID NO: 4 and the granzyme B portion may consist of SEQ ID NO: 9. The CSF1 portion may consist of SEQ ID NO: 5 and the granzyme B portion may consist of SEQ ID NO: 6. The CSF1 portion may consist of SEQ ID NO: 5 and the granzyme B portion may consist of SEQ ID NO: 7. The CSF1 portion may consist of SEQ ID NO: 5 and the granzyme B portion may consist of SEQ ID NO: 8. The CSF1 portion may consist of SEQ ID NO: 5 and the granzyme B portion may consist of SEQ ID NO: 9.

Any of I to VI above may comprise a leader sequence. When present, the leader sequence may be the most N-terminal component. Preferably, the leader sequence comprises SEQ ID NO: 12. More preferably, the leader sequence consists of SEQ ID NO: 12.

The exemplary fusion protein of I may, for example, comprise SEQ ID NO: 19 (GzmB-CSF) or SEQ ID NO: 28 (GzmB-CSF1 without a leader sequence).

The exemplary fusion protein of II may, for example, comprise SEQ ID NO: 15 (GzmB-CSF1-FL) or SEQ ID NO: 29 (GzmB-CSF1-FL without a leader sequence).

The exemplary fusion protein of III may, for example, comprise SEQ ID NO: 21 (GzmB-CSF1-FLFP without an epitope tag) or SEQ ID NO: 30 (GzmB-CSF1-FLFP without a leader sequence and an epitope tag). The exemplary fusion protein of III may, for example, comprise SEQ ID NO: 23 (GzmB-CSF1-FLFPlong without an epitope tag) or SEQ ID NO: 32 (GzmB-CSF1-FLFPlong without a leader sequence and an epitope tag).

The exemplary fusion protein of IV may, for example, comprise SEQ ID NO: 16 (GzmB-CSF1-FLFP) or SEQ ID NO: 25 (GzmB-CSF1-FLFP without a leader sequence). The exemplary fusion protein of IV may, for example, comprise SEQ ID NO: 18 (GzmB-CSF1-FLFPlong) or SEQ ID NO: 27 (GzmB-CSF1-FLFPling without a leader sequence).

The exemplary fusion protein of V may, for example, comprise SEQ ID NO: 22 (GzmB-CSF1-CL without an epitope tag) or SEQ ID NO: 31 (GzmB-CSF1-CL without a leader sequence and an epitope tag).

The exemplary fusion protein of VI may, for example, comprise SEQ ID NO: 17 (GzmB-CSF1-CL) or SEQ ID NO: 26 (GzmB-CSF1-CL without a leader sequence).

### Polynucleotide Sequence

Disclosed herein is a polynucleotide sequence encoding the fusion protein of the disclosure. The polynucleotide sequence may be codon optimised.

The polynucleotide sequence may comprise DNA. The polynucleotide sequence may comprise RNA. The polynucleotide sequence may comprise DNA and RNA. The polynucleotide sequence may consist of DNA. The polynucleotide sequence may consist of RNA. The polynucleotide sequence may consist of DNA and RNA.

The fusion protein encoded by the polynucleotide sequence may be any fusion protein described above. For instance, the polynucleotide sequence may encode any of the exemplary fusion proteins I to VI described above. Thus, the polynucleotide sequence may comprise (for instance, from 5′ to 3′):
I. A nucleic acid sequence encoding a granzyme B portion, a nucleic acid sequence encoding a flexible linker, and nucleic acid sequence encoding a CSF1 portion. The flexible linker may be (GGGGS)₄
II. A nucleic acid sequence encoding an epitope tag, a nucleic acid sequence encoding a granzyme B portion, a nucleic acid sequence encoding a flexible linker, and a nucleic acid sequence encoding a CSF1 portion. The epitope tag may be a FLAG-tag of SEQ ID NO: 10. The flexible linker may be (GGGGS)₄. The epitope tag may be a FLAG-tag of SEQ ID NO: 10 and the flexible linker may be (GGGGS)₄.
III. A nucleic acid sequence encoding a granzyme B portion, a nucleic acid sequence encoding a flexible linker, a nucleic acid sequence encoding a CSF1 portion, and a nucleic acid sequence encoding a fusogenic peptide. The flexible linker may be (GGGGS)₄. The fusogenic peptide may be a fusogenic peptide of SEQ ID NO: 11. The flexible linker may be (GGGGS)₄, and the fusogenic peptide may be a fusogenic peptide of SEQ ID NO: 11.
IV. A nucleic acid sequence encoding an epitope tag, a nucleic acid sequence encoding a granzyme B portion, a nucleic acid sequence encoding a flexible linker, a nucleic acid sequence encoding a CSF1 portion, and a nucleic acid sequence encoding a fusogenic peptide. The epitope tag may be a FLAG-tag of SEQ ID NO: 10. The flexible linker may be (GGGGS)₄. The fusogenic peptide may be a fusogenic peptide of SEQ ID NO: 11. The flexible linker may be (GGGGS)₄. The epitope tag may be a FLAG-tag of SEQ ID NO: 10, the flexible linker may be (GGGGS)₄, and the fusogenic peptide may be a fusogenic peptide of SEQ ID NO: 11.
V. A nucleic acid sequence encoding a granzyme B portion, a nucleic acid sequence encoding a cleavable linker, and a nucleic acid sequence encoding a CSF1 portion. The cleavable linker may comprise or consist of SEQ ID NO: 14. The cleavable linker may comprise or consist of SEQ ID NO: 33 or 34.
VI. A nucleic acid sequence encoding an epitope tag, a nucleic acid sequence encoding a granzyme B portion, a nucleic acid sequence encoding a cleavable linker, and a nucleic acid sequence encoding a CSF1 portion. The epitope tag may be a FLAG-tag of SEQ ID NO: 10. The cleavable linker may comprise or consist of SEQ ID NO: 14. The cleavable linker may comprise or consist of SEQ ID NO: 33 or 34. The epitope tag may be a FLAG-tag of SEQ ID NO: 10 and the cleavable linker may comprise or consist of SEQ ID NO: 14. The epitope tag may be a FLAG-tag of SEQ ID NO: 10 and the cleavable linker may comprise or consist of SEQ ID NO: 33 or 34.

Any of exemplary CSF1 portions and granzyme B portions described above in connection with exemplary fusion proteins may also apply to exemplary polynucleotide sequences.

Any of exemplary polynucleotide sequences I to VI above may comprise a nucleic acid sequence encoding a leader sequence. When present, the nucleic acid sequence encoding the leader sequence may be 5′ to the component first-mentioned in exemplary polynucleotide sequences I to VI. Preferably, the leader sequence comprises SEQ ID NO: 12. More preferably, the leader sequence consists of SEQ ID NO: 12.

Any of exemplary polynucleotide sequences I to VI above may comprise a Kozak consensus sequence. When present, the Kozak consensus sequence may be the most 5′ component.

### Virus

The disclosure provides a virus that comprises the polynucleotide sequence disclosed herein. Any of the aspects described above in connection with the polynucleotide sequence or fusion protein may also apply to the virus of the disclosure.

The virus may be used to deliver the fusion protein to an individual. For instance, the virus may be used to administer the fusion protein to an individual in which the fusion protein is expected to have therapeutic benefit. For example, the virus may be used to deliver the fusion protein to an individual having AML or another type of cancer such as a solid tumor. The virus may be used to deliver the fusion protein to an individual having an inflammatory disease. In other words, the virus may be used as a delivery vector.

The virus may, for example, be a lentivirus, a retrovirus, an adenovirus, an adeno-associated virus (AAV), a vaccinia virus or a herpes simplex virus. Preferably, the virus is an adenovirus.

The virus may have oncolytic activity. In other words, the virus may be an oncolytic virus. For instance, the virus may be an oncolytic adenovirus. Oncolytic activity may confer the virus with an ability to mount two-pronged attack against cancer cells. Firstly, oncolytic activity directly destroys cancer cells. Secondly, delivery of the polynucleotide leads to expression of the fusion protein, which leads to cytotoxicity in cells that express CSF1 receptor. In this way, bystander CSF1 receptor-expressing cells can be killed even though they are not infected with the virus. Bystander cells may comprise cancer cells, such as AML cells. Bystander cells may comprise TAMs, such as immunosuppressive TAMs. Killing of TAMs may reduce immunosuppression in the tumour microenvironment and thus promote anti-tumour immune responses.

The virus may comprise a targeting molecule to ensure efficient transduction with the polynucleotide sequence encoding the fusion protein of the disclosure. The targeting molecule will typically be provided wholly or partly on the surface of the virus in order for the molecule to be able to direct the virus to target cells. The target cell for AML may be a myeloblast or a leukaemic cell. The target cell for a solid tumour may be a cancer cell, or a non-cancer cell in the tumour microenvironment. The non-cancer cell in the tumour microenvironment may, for example, be an immune cell such as a T cell, a B cell, a NK cell, a NKT cell, a monocyte, or a macrophage. The target cell for an inflammatory disease may, for example, be a monocyte or macrophage.

### T Cell

The disclosure provides a T cell comprising the polynucleotide sequence described herein. Any of the aspects described above in connection with the polynucleotide sequence or fusion protein may also apply to the T cell of the disclosure.

The T cell may, for example, express CD4. In other words, the T cell may be a CD4+ T cell. The CD4+ T cell may, for example, be a helper T cell. The T cell may, for example, express CD8. In other words, the T cell may be a CD8+ T cell. The CD8+ T cell may, for example, be a cytotoxic T cell. The T cell may express both CD4 and CD8.

The T cell may, for example, be a CAR T cell. That is, the T cell may express a chimeric antigen receptor (CAR). CARs of various specificities are well-known in the art. The CAR may be specific for any desired antigen. For example, the CAR may bind to a tumour specific antigen (TSA) or tumour associated antigen (TAA). In other words, the CAR may be specific for or recognise a TSA or TAA. The TSA may be an antigen that is specific for AML. The TSA may be an antigen that is specific for a solid tumour. The TAA may be an antigen that is associated with AML. The TAA may be an antigen that is associated with a solid tumour. In any case, the solid tumour may be a tumour of the anal cancer, bile duct (cholangiocarcinoma), bladder, bone, bowel, brain, breast, colon, rectum, cervix, endocrine system, eye (such as ocular melanoma), fallopian tube, gall bladder, head and/or neck, kidney, larynx, liver, lung, lymphocytes (lymphoma) lymph node, mesothelium, neuroendocrine system, ovary, oesophagus, pancreas, penis, peritoneum, prostate, skin, small bowel, spinal cord, stomach, testes, thymus, thyroid, trachea, vagina, vulva or endometrium. The solid tumour may, for instance, be a glioma.

Preferably, the T cell expresses the fusion protein encoded by the polynucleotide. For example, the T cell may constitutively express the fusion protein encoded by the polynucleotide. Alternatively, the T cell may inducibly express the fusion protein encoded by the polynucleotide. For instance, expression of the fusion protein may be induced following binding of cognate antigen to an antigen receptor comprised in the T cell. For example, the T cell may be a CAR T cell as described above, and binding of cognate antigen to the CAR may induces expression of the fusion protein encoded by the polynucleotide.

In order to induce expression of the fusion protein encoded by the polynucleotide, the CAR may be a synthetic notch (SynNotch) receptor. SynNotch receptors are well-known in the art. Typically, a SynNotch receptor comprises an extracellular antigen-binding domain, a Notch core regulatory region, and an intracellular domain. Binding of the extracellular domain to cognate antigen leads to conformational changes in the Notch core regulatory region that in turn lead to release of the intracellular domain. For instance, the conformational changes may expose sites for proteolytic cleavage, and cleavage may release the intracellular domain. Typically, the intracellular domain is a transcription factor. The polynucleotide encoding the fusion protein may be operably linked to a promoter that is activatable by the transcription factor.

The T cell may, for example, be autologous with respect to an individual into which it is to be administered. The T cell may, for example, be allogeneic with respect to an individual into which it is to be administered.

Expression of the fusion protein of the disclosure by the T cell may confer the T cell with an ability to mount two-pronged attack against target cells, such as cancer cells. Firstly, the T cell itself may contribute to an immune response against the target cells. For instance, the T cell may be a cytotoxic T cell that destroys target cells. The T cell may, for example, be a helper T cell that is activated by target cells and assists in a cell-mediated or humoral immune response against the target cells. Secondly, the fusion protein expressed by the T cell is cytotoxic to cells that express CSF1 receptor. Thus, the fusion protein may kill CSF1 receptor-expressing target cells, such as AML cells or inflammatory cells (e.g. monocytes or macrophages). The fusion protein may kill CSF1 receptor-expressing bystander cells, such as TAMs. Killing of TAMs may, for example, reduce immunosuppression in the tumour microenvironment and thus promote anti-tumour immune responses.

### Medicaments and Medical Uses

Disclosed herein is a method of treating a disease in an individual, comprising administering to the individual the fusion protein, polynucleotide, virus or the T cell of the disclosure. The method may further comprise administering to the individual an additional therapeutic agent.

Also disclosed herein is the individual the fusion protein, polynucleotide, virus or the T cell of the disclosure for use in a method of treating a disease in an individual, the method comprising administering the fusion protein, the polynucleotide sequence, the virus or the T cell to the individual. The method may further comprise administering to the individual an additional therapeutic agent.

Further disclosed here in is use of the fusion protein, polynucleotide, virus or the T cell of the disclosure in the manufacture of a medicament for treating a disease in an individual.

Any of the aspects described above in connection with the fusion protein of the disclosure may also apply to the method of treatment or medical use of the disclosure. Any of the aspects described above in connection with the polynucleotide of the disclosure may also apply to the method of treatment or medical use of the disclosure. Any of the aspects described above in connection with the virus of the disclosure may also apply to the method of treatment or medical use of the disclosure. Any of the aspects described above in connection with the T cell of the disclosure may also apply to the method of treatment or medical use of the disclosure.

### Disease

The disease may be any disease that may benefit from destruction of CSF1 receptor-expressing cells.

The disease may, for instance, be cancer. The cancer may, for example, be a cancer in which cancer cells express CSF1 receptor. In other words, the cancer may be a CSF1 receptor-expressing cancer. The CSF1 receptor-expressing cancer may, for instance, be a CSF1 receptor-expressing haematolgic malignancy, such as a leukaemia. For example, the CSF1 receptor-expressing cancer may be AML.

The cancer, may, for example, be cancer in which CSF1 receptor-expressing cells promote the development or progression of disease. For instance, the cancer may be a cancer in which CSF1 receptor-expressing cells suppress an anti-cancer immune response. For example, the cancer may be a cancer in which CSF1 receptor-expressing cells promote an immunosuppressive tumour microenvironment. For example, the cancer may be a cancer in which TAMs promote an immunosuppressive tumour microenvironment, such as a solid tumour. The solid tumour may be any solid tumour described above or below.

The cancer may, for example, be a solid tumour. For instance, the cancer may be a solid tumour that comprises cells that express CSF1 receptor. The cells that express CSF1 receptor may, for instance, be TAMs. The TAMs may, for, example, be immunosuppressive TAMs. The role of immunosuppressive TAMs is well known in many solid tumours. The solid tumour may, for instance, be a tumour of the anal cancer, bile duct (cholangiocarcinoma), bladder, bone, bowel, brain, breast, colon, rectum, cervix, endocrine system, eye (such as ocular melanoma), fallopian tube, gall bladder, head and/or neck, kidney, larynx, liver, lung, lymphocytes (lymphoma) lymph node, mesothelium, neuroendocrine system, ovary, oesophagus, pancreas, penis, peritoneum, prostate, skin, small bowel, spinal cord, stomach, testes, thymus, thyroid, trachea, vagina, vulva or endometrium. The solid tumour may, for instance, be a glioma.

The disease may, for example, be an inflammatory disease. The inflammatory disease may, for instance, be an inflammatory disease in which CSF1 receptor-expressing cells contribute to inflammation. For example, the inflammatory disease may be an inflammatory disease in which monocytes and/or macrophages contribute to inflammation. In other words, the pathogenesis of the inflammatory disease may be mediated by macrophages and/or monocytes, in whole or in part. Macrophages and/or monocytes may, for example, contribute to the development or progression of clinical signs or symptoms. The inflammatory disease may, for example, be an allergy. The inflammatory disease may, for example, be an autoimmune disease, such as coeliac disease. The inflammatory disease may, for example, be asthma. The inflammatory disease may, for example, be glomerulonephritis, hepatitis or inflammatory bowel disease. The inflammatory disease may, for example, be reperfusion injury. The inflammatory disease may, for example, be transplant rejection or graft-verus-host disease (GvHD). The inflammatory disease may, for example, be atherosclerosis.

The disease may, for example, be an infectious disease. The infectious disease may, for instance, be an infectious disease in which CSF1 receptor-expressing cells contribute to pathogenesis. For example, the infectious disease may be an infectious disease in which monocytes and/or macrophages contribute to pathogenesis. In other words, the pathogenesis of the infectious disease may be mediated by macrophages and/or monocytes, in whole or in part. Macrophages and/or monocytes may, for example, contribute to the development or progression of clinical signs or symptoms. The infectious disease may, for example, be a bacterial infection. For example, the infectious disease may, for example, be Whipple's disease, which is caused by the bacterium Tropheryma whipplei. The infectious disease may, for example, be a viral infection. For example, the infectious disease may, for example, be COVID-19, which is caused by the virus SARS coronavirus 2 (SARS-CoV-2). The infectious disease may, for example, be a protozoal infection.

### Individual

The individual may, for example, be any individual that may benefit from destruction of CSF1 receptor-expressing cells. For instance, the individual may have any of the diseases described above. The individual may be suspected of having any of the diseases described above.

The individual may, for example, be a mammal. Preferably, the mammal is a human.

The individual may be of any age. For example, the individual may be a juvenile. The individual may, for example, be an adult.

### Administration

The fusion protein, polynucleotide, virus or the T cell may be administered by any route. Suitable routes include, but are not limited to, the intravenous, intrathecal, intracerebral ventricular, intramuscular, intraperitoneal, subcutaneous, intradermal, transdermal and oral/buccal routes.

The fusion protein, polynucleotide, virus or the T cell may be comprised in a composition that comprises a physiologically acceptable carrier or diluent. Typically, such compositions are prepared as liquid suspensions of the fusion protein, polynucleotide, virus or the T cell. The fusion protein, polynucleotide, virus or the T cell may be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions may contain minor amounts of auxiliary substances such as wetting or emulsifying agents, and/or pH buffering agents.

The fusion protein, polynucleotide, virus or the T cell is administered in a manner compatible with the dosage formulation and in such amount will be therapeutically effective. The quantity to be administered depends, for example, on the subject to be treated, the nature of the disease (e.g. type of cancer), and so on. Precise amounts of the fusion protein, polynucleotide, virus or the T cell required to be administered may depend on the judgement of the practitioner and may be peculiar to each subject.

### Further Therapeutic Agent

As set out above, the method may further comprise administering to the individual a further therapeutic agent. Accordingly, the fusion protein, polynucleotide, virus or the T cell of the disclosure may be used in combination therapy.

The additional therapeutic agent may be administered to the individual at the same time as the fusion protein, polynucleotide, virus or the T cell of the disclosure. In this case, the additional therapeutic agent may be comprised in the same composition as the fusion protein, polynucleotide, virus or the T cell of the disclosure.

The additional therapeutic agent may be administered to the individual at a different time from the fusion protein, polynucleotide, virus or the T cell of the disclosure. In this case, the additional therapeutic agent may be comprised in a different composition from the fusion protein, polynucleotide, virus or the T cell of the disclosure. The additional therapeutic agent may, for example, be administered to the individual before the fusion protein, polynucleotide, virus or the T cell of the disclosure. The additional therapeutic agent may, for example, be administered to the individual after the fusion protein, polynucleotide, virus or the T cell of the disclosure.

The additional therapeutic agent may comprise a small molecule. The additional therapeutic agent may, for example, comprise a chemotherapeutic. The chemotherapeutic may, for example, comprise carboplatin, temozolomide, lomustine, and/or irinotecan. The additional therapeutic agent may, for example, comprise a inhibitor such as a kinase inhibitor. The kinase inhibitor may, for example, comprise regorafenib. The additional therapeutic may, for example comprise a monoclonal antibody. The monoclonal antibody may, for example, comprise bevacizumab. The additional therapeutic agent may, for example, comprise radiotherapy. When the disease is a cancer (such as a solid tumour), the additional therapeutic agent may comprise a known treatment for that cancer. For example, when the solid tumor is glioblastoma, the additional therapeutic agent may, for example, comprise carboplatin, temozolomide, lomustine, irinotecan, regorafenib, bevacizumab and/or radiotherapy.

The additional therapeutic agent may, for example, comprise an oncolytic virus. For instance, when the disease is a cancer, the additional therapeutic agent may comprise an oncolytic virus that is effective against that cancer. For example, when the disease is a solid tumour, the additional therapeutic agent may comprise an oncolytic virus that is effective against that solid tumour. For instance, when the disease is a glioma, the additional therapeutic agent may comprise an oncolytic virus that is effective against that glioma.

The additional therapeutic agent may, for example, comprise a CAR T cell. The CAR T cell may, for example, express a CAR specific for an antigen expressed by target cells. For instance, when the disease is a cancer, the CAR T cell may express a CAR specific for a TAA or TSA expressed by that cancer. When the disease is a solid tumour, the CAR T cell may express a CAR specific for a TAA or TSA expressed by that solid tumour. when the disease is a glioma, the CAR T cell may express a CAR specific for a TAA or TSA expressed by that glioma.

The additional therapeutic agent may, for example, target cells that express CSF1 receptor. Thus, the additional therapeutic agent may promote treatment of AML. The effect of the additional therapeutic agent may, for example, be additive to or synergistic with the effect of the fusion protein, polynucleotide, virus or the T cell of the disclosure.

The additional therapeutic agent may, for example, target TAMs. The additional therapeutic agent may therefore reduce immunosuppression in the tumour microenvironment and thus promote anti-tumour immune responses. In this way, the additional therapeutic agent may promote treatment of solid tumours. The effect of the additional therapeutic agent may, for example, be additive to or synergistic with the effect of the fusion protein, polynucleotide, virus or the T cell of the disclosure.

The additional therapeutic agent may, for example, target CSF1 receptor-expressing macrophages and/or monocytes involved in disease pathogenesis. In this way, the additional therapeutic agent may promote treatment of inflammatory disease. The effect of the additional therapeutic agent may, for example, be additive to or synergistic with the effect of the fusion protein, polynucleotide, virus or the T cell of the disclosure.

The following Examples illustrate the invention.

### EXAMPLES

### Example 1

### Overview

The inventors evaluated whether fusion protein-encoding pTRPE expression vectors could produce and secrete functional proteins.

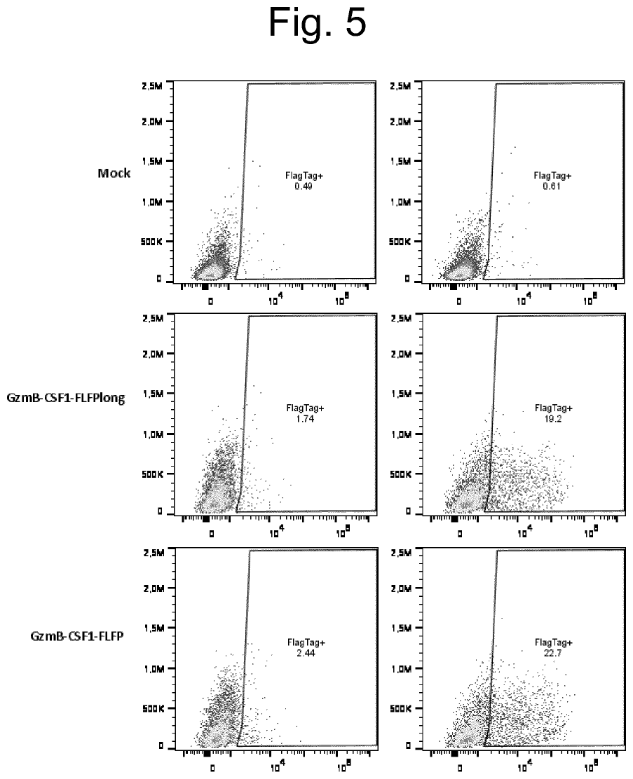

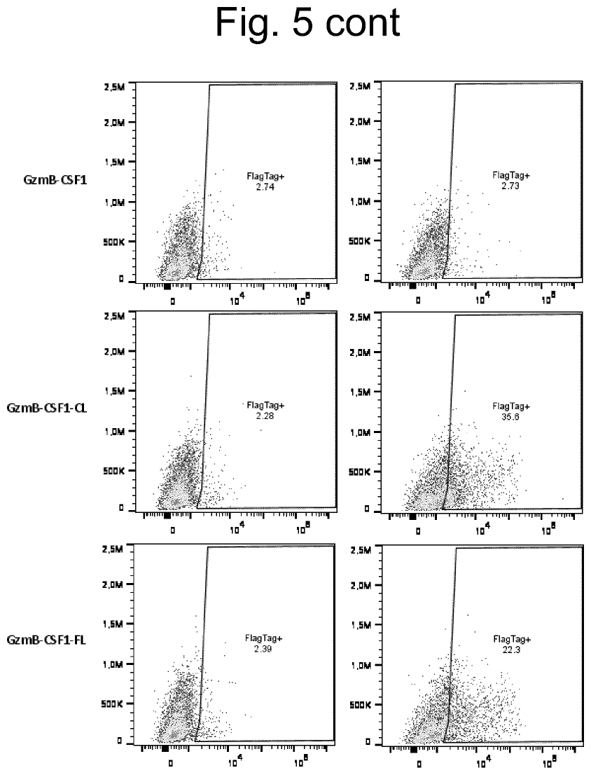

To assess the production of the proteins, HEK293 were transfected with mock or pTRPE-fusion proteins, followed by intracellular FLAG tag detection. Fusion proteins were detected in all HEK293 cells transfected with FLAG tag-labelled versions (FIG. 5).

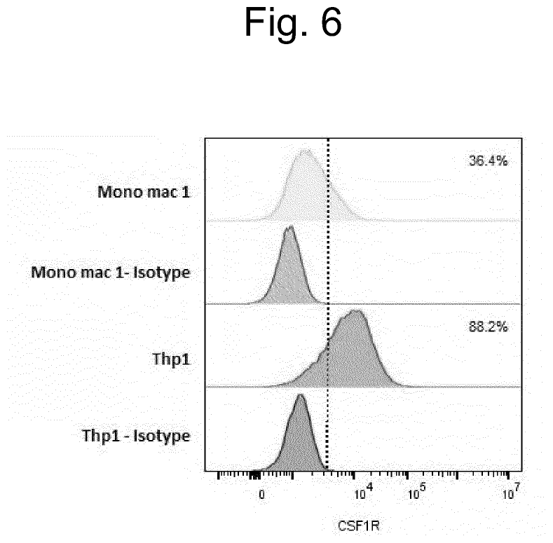

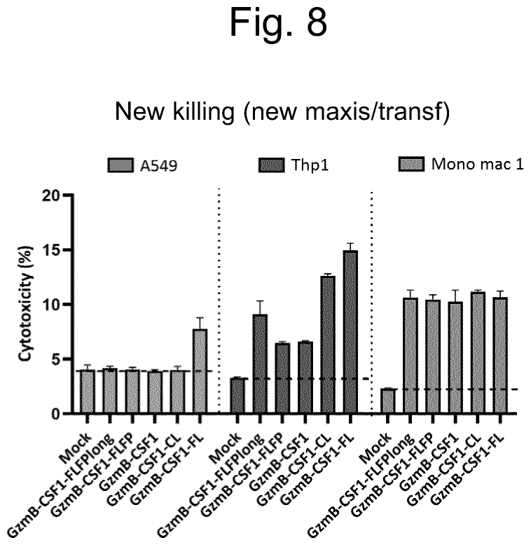

Next, flow cytometry-based fusion protein-mediated cytotoxicity assays were performed by culturing A549 (5% CSF1R+), Mono mac 1 (36% CSF1R+), and Thp1 (88% CSF1R+) cell lines (FIG. 6) with non-concentrated concentrated supernatants of HEK293 transfected cells. Slight cytotoxicity of Thp1 cells were observed when cocultured with non-concentrated supernatants (FIG. 8).

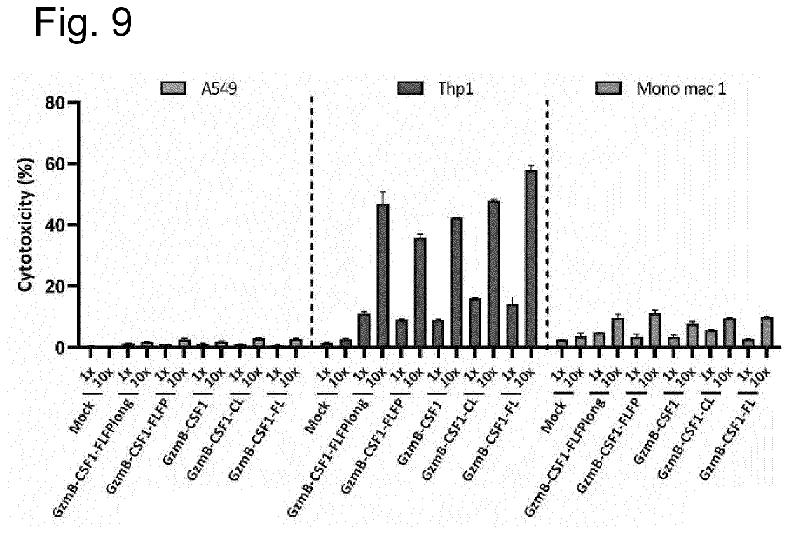

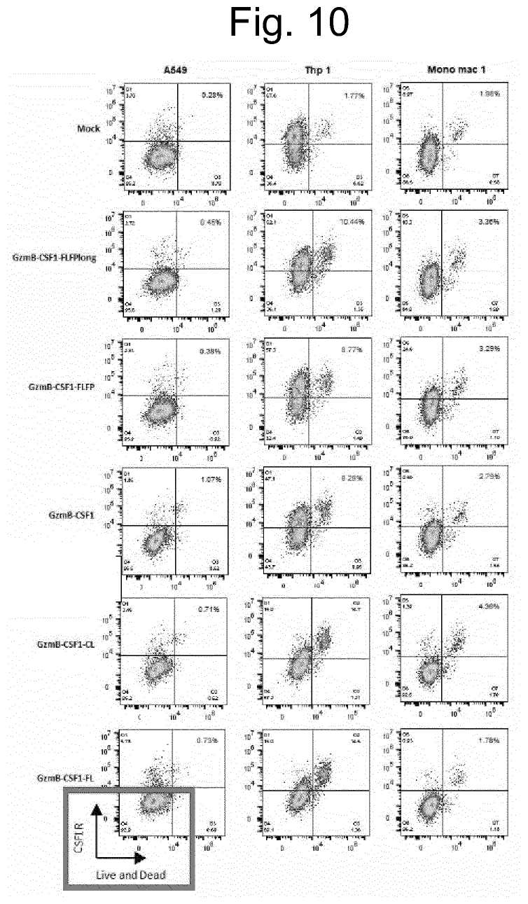

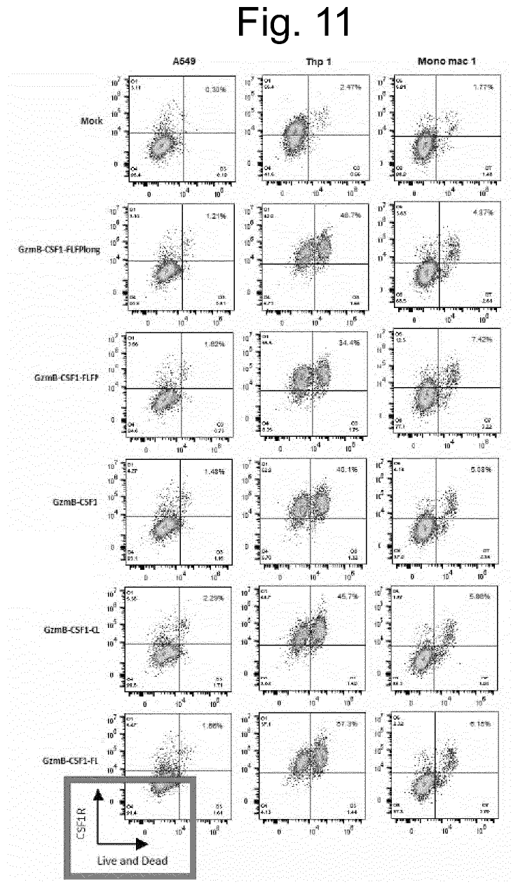

The inventors next performed a cytotoxicity assay with non-concentrated supernatants (1×) and 10×-concentrated supernatants. Increased cytotoxicity was observed in both Mono mac 1 and Thp1 cells when incubated with 10× concentrated supernatants (FIG. 9). In addition, FIGS. 10 and 11 show specific killing of CSF1R-positive cells in both Mono mac 1 and Thp1 cultures. Importantly, cytotoxicity of A549 cells was nearly undetectable, which accords with its 5% CSF1R expression. GzmB-CSF1-FL and GzmB-CSF1-CL versions showed best killing capacity.

### Design of Fusion Proteins

The following CSF1-granzyme B fusion proteins were designed:
GzmB-CSF1-FL (SEQ ID NO: 15). GzmB-CSF1-FL comprises a flexible linker between the granzyme B portion and the CSF1 portion. GzmB-CSF1-FL is encoded by a polynucleotide sequence that comprises, 5′ to 3′:
kozac-leader seq-FLAG-tag-gzmB-(GGGGS)₄-CSF1-stop+polyA

The epitope tag (FLAG-tag) is ultimately cleaved from the fusion protein, giving rise to SEQ ID NO: 20. SEQ ID NO: 24 relates to a form of GzmB-CSF1-FL without the leader sequence. SEQ ID NO: 29 relates to a form of GzmB-CSF1-FL without the leader sequence and the epitope tag.
GzmB-CSF1-FLFP (SEQ ID NO: 16). GzmB-CSF1-FLFP comprises a flexible linker between the granzyme B portion and the CSF1 portion. GzmB-CSF1-FLFP also comprises a fusogenic peptide (FP) to help it to escape from endosomes. The CSF1 portion comprises residues 33 to 181 of CSF1. In other words, the CSF1 portion comprises SEQ ID NO: 2. GzmB-CSF1-FLFP is encoded by a polynucleotide sequence that comprises, 5′ to 3′:
kozac-leader seq-FLAG-tag-gzmB-(GGGGS)₄-CSF1 (33-181)-fusogenic peptide-stop+poly A

The epitope tag (FLAG-tag) is ultimately cleaved from the fusion protein, giving rise to SEQ ID NO: 21. SEQ ID NO: 25 relates to a form of GzmB-CSF1-FLFP without the leader sequence. SEQ ID NO: 30 relates to a form of GzmB-CSF1-FLFP without the leader sequence and the epitope tag.
GzmB-CSF1-CL (SEQ ID NO: 17). GzmB-CSF1-CL comprises a cleavable linker between the granzyme B portion and the CSF1 portion. GzmB-CSF1-CL is encoded by a polynucleotide sequence that comprises, 5′ to 3′:
kozac-leader seq-FLAG-tag-gzmB-GGGGS-LRMKLPKP-GGGGS-CSF1 stop+polyA

The epitope tag (FLAG-tag) is ultimately cleaved from the fusion protein, giving rise to SEQ ID NO: 22. SEQ ID NO: 26 relates to a form of GzmB-CSF1-CL without the leader sequence. SEQ ID NO: 31 relates to a form of GzmB-CSF1-CL without the leader sequence and the epitope tag.

An alternative cleavable linker may be used. The alternative cleavable linker may, for example, comprise or consist of SEQ ID NO: 34. In this case, the fusion protein may be represented by SEQ ID NO: 36. The epitope tag (FLAG-tag) is ultimately cleaved from the fusion protein, giving rise to SEQ ID NO: 37. SEQ ID NO: 38 relates to a form of GzmB-CSF1-CL without the leader sequence. SEQ ID NO: 39 relates to a form of GzmB-CSF1-CL without the leader sequence and the epitope tag.
GzmB-CSF1-FLFPlong (SEQ ID NO: 18). GzmB-CSF1-FLFPlong comprises a flexible linker between the granzyme B portion and the CSF1 portion. GzmB-CSF1-FLFPlong also comprises a fusogenic peptide (FP) to help it to escape from endosomes. The CSF1 portion comprises residues 33 to 196 of CSF1. In other words, the CSF1 portion comprises SEQ ID NO: 3. GzmB-CSF1-FLFPlong is encoded by a polynucleotide sequence that comprises, 5′ to 3′:
kozac-leader seq-FLAG-tag-gzmB-(GGGGS)₄-CSF1 (33-196)-stop+polyA

The epitope tag (FLAG-tag) is ultimately cleaved from the fusion protein, giving rise to SEQ ID NO: 23. SEQ ID NO: 27 relates to a form of GzmB-CSF1-FLFPlong without the leader sequence. SEQ ID NO: 32 relates to a form of GzmB-CSF1-FLFPlong without the leader sequence and the epitope tag.
GzmB-CSF1 (SEQ ID NO: 19). GzmB-CSF1 comprises a flexible linker between the granzyme B portion and the CSF1 portion. GzmB-CSF1 does not comprise an eptiotpe tag, such as a FLAG-tag. GzmB-CSF1 is encoded by a polynucleotide sequence that comprises, 5′ to 3′:
kozac-leader seq-gzmB-(GGGGS)₄-CSF1-stop+polyA

SEQ ID NO: 28 relates to a form of GzmB-CSF1 without the leader sequence.

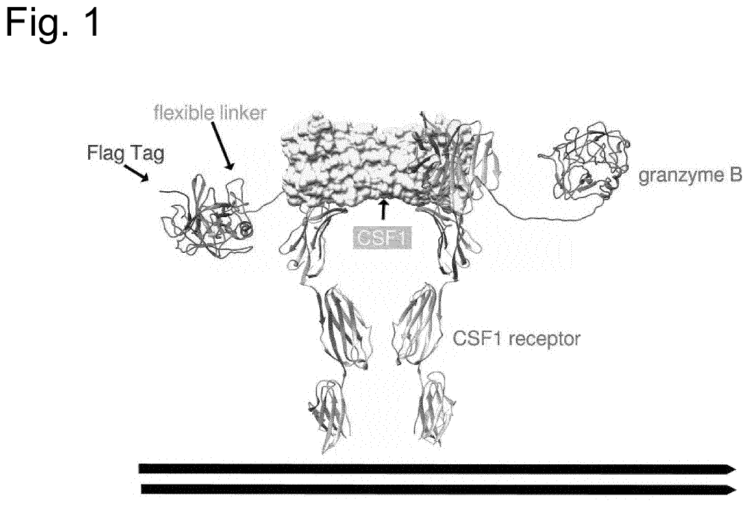

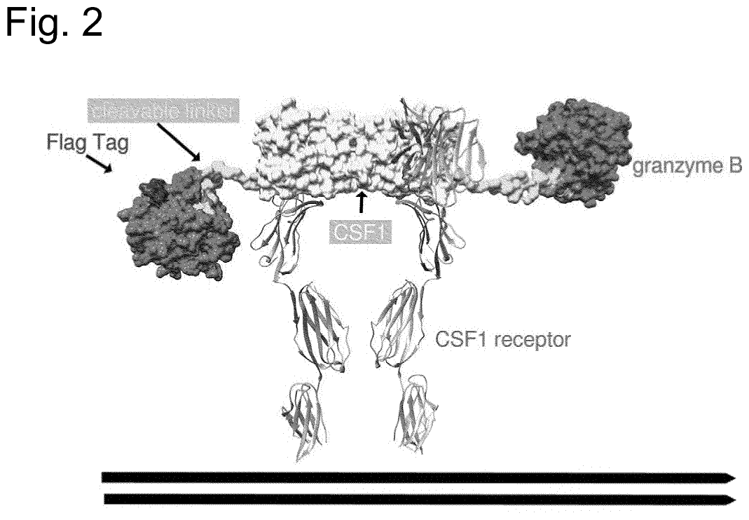

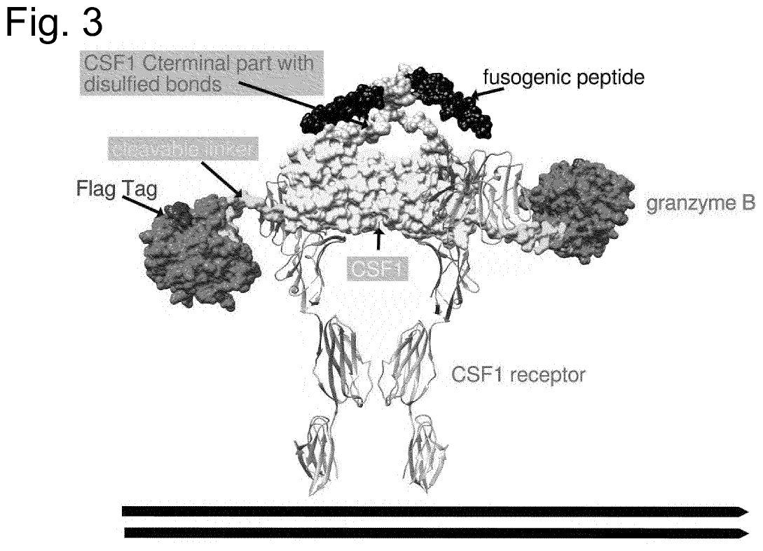

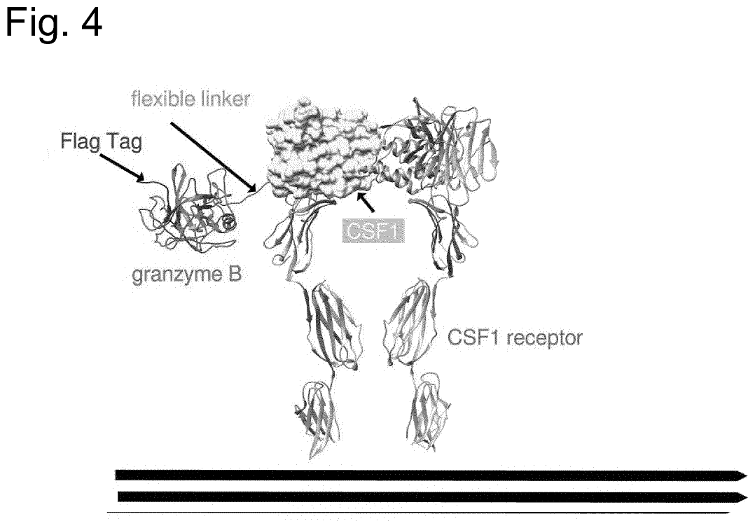

FIGS. 1, 2, 3, and 4 provide a 3D representation of different GzmB-CSF1 fusion proteins.

### Transfection of HEK293 Cells

To determine expression of GzmB-CSF1 fusion proteins by HEK293 cells, HEK293 were transfected with mock or the indicated pTRPE-GzmB-CSF1 versions (GzmB-CSF1-FL (SEQ ID NO: 15), GzmB-CSF1-FLFP (SEQ ID NO: 16), GzmB-CSF1-CL (SEQ ID NO: 17), GzmB-CSF1-FLFPlong (SEQ ID NO: 18), or GzmB-CSF1 (SEQ ID NO: 19)). After 72h, GolgiSTOP was added to the cells, incubated 4h, and intracellular protein expression was detected by flow cytometry incubating cells with a FITC-conjugated anti-FLAG antibody. Results are shown in FIG. 5.

### CSF1 Receptor Expression

CSF1 receptor (CSF1R) expression was determined for Mono mac 1 and Thp1 cells. The cells were evaluated for CSF1R expression by flow cytometry. Cells were incubated with an APC-coupled anti-CSF1R antibody or its corresponding isotype control. The results are shown in FIG. 6. 36.4% of MonoMac1 cells and 88.2% of THP1 cells expressed CSF1R at a level above that of the isotype control.

### Cytotoxicity Assay

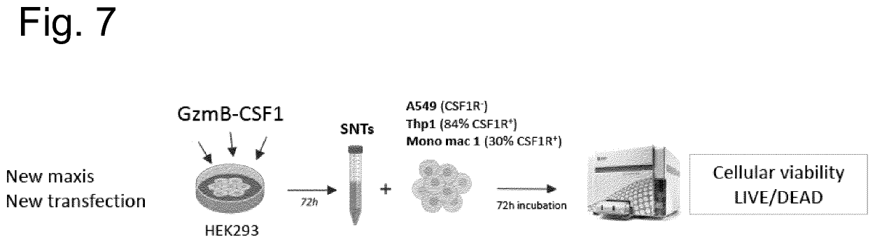

The ability of GzmB-CSF1-FL, GzmB-CSF1-FLFP, GzmB-CSF1-CL, GzmB-CSF1-FLFPlong and GzmB-CSF1 to induce cytotoxicity in CSF1R-expressing cells was assayed in vitro. A schema of the method is provided in FIG. 7. HEK293 were transfected with the different pTRPE-GzmB-CSF1 versions. After 72h, supernatants (SNTs) were collected and incubated with A549 (5% CSF1R-positive cells), Thp1 (84% CSF1R-posivite cells), and Mono mac 1 (30% CSF1R-positive cells) for 72h. Cellular viability was measured by flow cytometry after LIVE/DEAD staining.

FIG. 8 shows cytotoxicity of GzmB-CSF1 fusion proteins-containing supernatants of HEK293 transfected cells, according to a first assay. A549, Mono mac 1, and Thp1 cells were incubated with non-concentrated supernatants for 72 hours. Cell viability was measured by flow cytometry after LIVE/DEAD staining. Mean values±SD are plotted (n=3).

FIG. 9 shows cytotoxicity of GzmB-CSF1 fusion proteins-containing supernatants of HEK293 transfected cells, according to a second assay. A549, Mono mac 1, and Thp1 cells were incubated with non-concentrated (1×) or 10× concentrated (10×) supernatants for 72 hours. Cell viability was measured by flow cytometry after LIVE/DEAD staining. Mean values±SD are plotted (n=3). FIG. 10 provides a plot representation of the cytotoxicity assay of FIG. 9 with non-concentrated supernatants. FIG. 11 provides a plot representation of the cytotoxicity assay FIG. 9 with 10× concentrated supernatants.

### Example 2

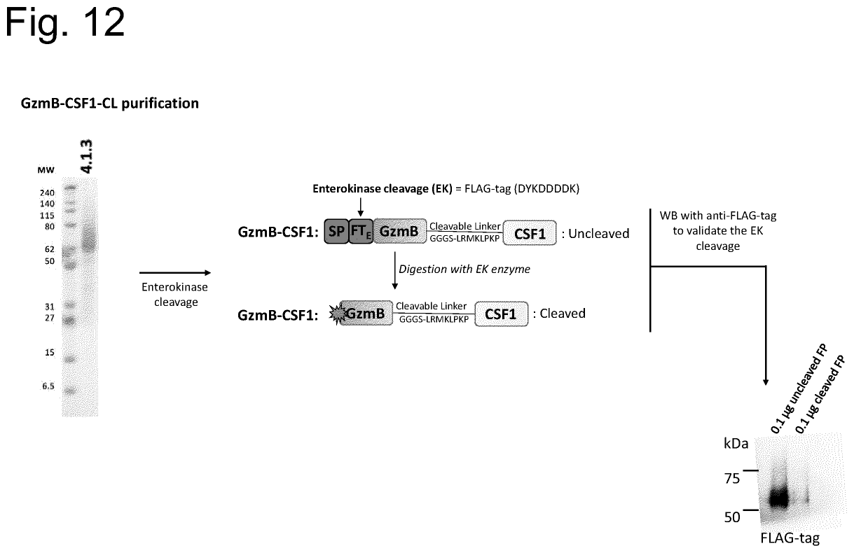

GzmB-CSF1-CL fusion protein was digested with an Enterokinase enzyme to obtain an active GzmB-CSF1-CL protein. The enterokinase cleavage site is DYKDDDDK, which corresponds to the FLAG-tag sequence on the GzmB-CSF1-CL fusion protein. A Western Blot was performed after digestion to detect the loss of FLAG-tag on digested GzmB-CSF1-CL fusion protein. Results are shown in FIG. 12.

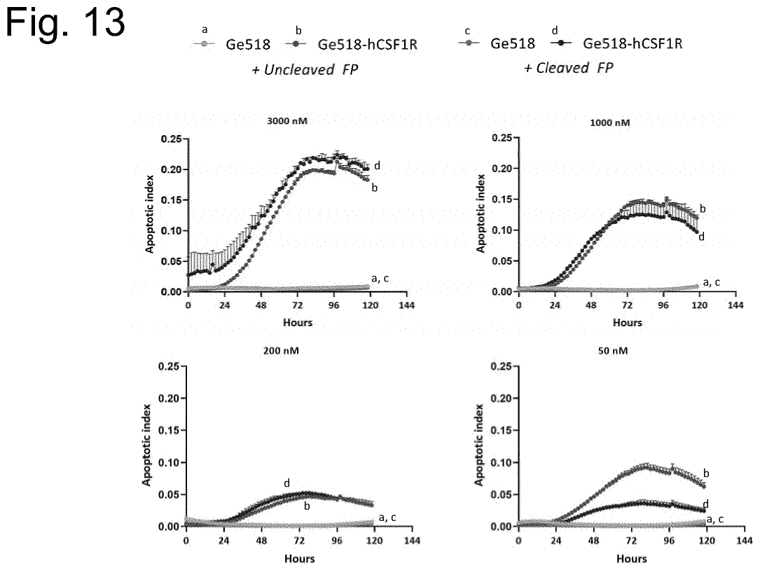

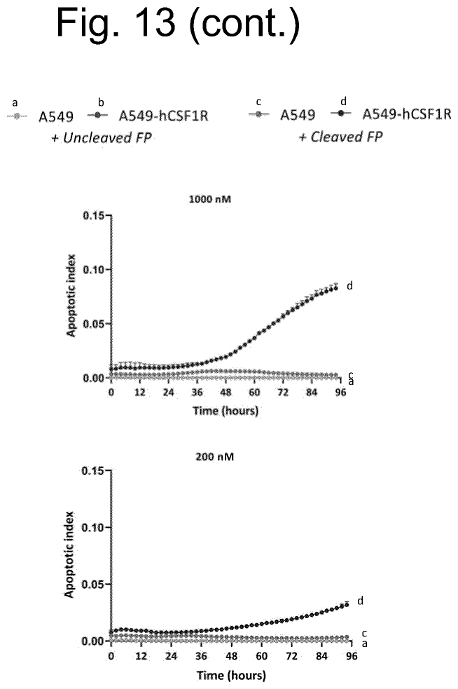

Cleaved and uncleaved GzmB-CSF1-CL was used to compare cytotoxicity capacity and specificity on different hCSF1R-positive and negative cell lines. In particular, the glioblastoma patient derived-cell line Ge518 and the lung cancer cell line A549 were transduced with a lentivirus to express the human CSF1R on the membrane (Ge518-hCSF1R and A549-h CSF1R, respectively). Cells were cocultured with different concentrations of the cleaved or uncleaved GzmB-CSF1-CL fusion protein, and the cytotoxicity was evaluated over time using the Incucyte machine. Data in FIG. 13 represent mean±SD of apoptotic index (cell death/confluence).

1. A fusion protein comprising a colony stimulating factor 1 (CSF1) portion and a granzyme B portion.

2. The fusion protein of claim 1, wherein:
(a) the CSF1 portion comprises the sequence of SEQ ID NO: 2, SEQ ID NO: 3, SEQ ID NO: 4; and/or
(b) the granzyme B portion comprises the sequence of SEQ ID NO: 7, SEQ ID NO: 8, or SEQ ID NO: 9.

3. The fusion protein of claim 1 or 2, further comprising:
(a) an epitope tag, optionally wherein the epitope tag is a FLAG-tag, and optionally wherein the FLAG-tag comprises the sequence of SEQ ID NO: 10; and/or
(b) a fusogenic peptide, optionally wherein the fusogenic peptide comprises the sequence of SEQ ID NO: 11; and/or
(c) a leader sequence, optionally wherein the leader sequence comprises the sequence of SEQ ID NO: 12.

4. The fusion protein of any one of the preceding claims, further comprising a linker, optionally wherein the linker links the CSF1 portion to the granzyme B portion.

5. The fusion protein of claim 4, wherein:
(a) the linker is a flexible linker, optionally wherein the flexible linker comprises the sequence of SEQ ID NO: 13; or
(b) the linker is a cleavable linker, optionally wherein the cleavable linker comprises the sequence of SEQ ID NO: 14, 33 or 34.

6. The fusion protein of any one of the preceding claims, wherein the fusion protein comprises the sequence of:
(a) any one of SEQ ID NO: 15, SEQ ID NO: 16, SEQ ID NO: 17, SEQ ID NO: 18, SEQ ID NO: 19 and SEQ ID NO: 36; or
(b) any one of SEQ ID NO: 20, SEQ ID NO: 21, SEQ ID NO: 22, SEQ ID NO: 23 and SEQ ID NO: 37; or
(c) any one of SEQ ID NO: 24, SEQ ID NO: 25, SEQ ID NO: 26, SEQ ID NO: 27, SEQ ID NO: 28 and SEQ ID NO: 38; or
(d) any one of SEQ ID NO: 29, SEQ ID NO: 30, SEQ ID NO: 31, SEQ ID NO: 32 and SEQ ID NO: 39.

7. A polynucleotide sequence encoding the fusion protein of any one of claims 1 to 6.

8. A virus comprising the polynucleotide sequence of claim 7, optionally wherein the virus is an oncolytic virus, an adenovirus, or an oncolytic adenovirus.

9. A T cell comprising the polynucleotide sequence of claim 7 and optionally expressing a chimeric antigen receptor (CAR), optionally wherein the CAR binds to a tumour specific antigen (TSA) or tumour associated antigen (TAA).

10. The T cell of claim 9, wherein:
(a) the T cell constitutively expresses the fusion protein encoded by the polynucleotide; or
(b) binding of cognate antigen to the CAR induces expression of the fusion protein encoded by the polynucleotide, optionally wherein the CAR is a synthetic notch (SynNotch) receptor.

11. A method of treating a disease in an individual, comprising administering to the individual (i) the fusion protein of any one of claims 1 to 6, the polynucleotide sequence of claim 7, the virus of claim 8, or the T cell of claim 9 or 10, and optionally (ii) an additional therapeutic agent.

12. The fusion protein of any one of claims 1 to 6, the polynucleotide sequence of claim 7, the virus of claim 8, or the T cell of claim 9 or 10 for use in a method of treating a disease in an individual, the method comprising administering to the individual (i) the fusion protein, the polynucleotide sequence, the virus or the T cell, and optionally (ii) an additional therapeutic agent.

13. The method of claim 11, or the fusion protein, polynucleotide sequence, virus or T cell for use of claim 12, wherein the disease is cancer, optionally wherein (a) the cancer is a cancer in which cancer cells express CSF1 receptor, optionally wherein the cancer is acute myeloid leukemia (AML); or
(b) the cancer is a solid tumour, optionally wherein the solid tumour comprises cells that express CSF1 receptor.

14. The method of claim 13 (b), or the fusion protein, polynucleotide sequence, virus or T cell for use of claim 13 (b), wherein:
(i) the cells that express CSF1 receptor are tumour infiltrating macrophages (TAMs); and/or
(ii) the solid tumour is a glioma.

15. The method of claim 11, or the fusion protein, polynucleotide sequence, virus or T cell for use of claim 12, wherein the disease is an inflammatory disease or infectious disease, optionally wherein:
(a) the pathogenesis of the inflammatory disease or infectious disease is mediated by macrophages and/or monocytes; and/or
(b) the inflammatory disease is Whipple's disease or the infectious disease is COVID-19.


SEQUENCE LISTING (39 sequences)

SEQ ID NO: 1 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 2 - protein, 149 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 3 - protein, 164 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKA

SEQ ID NO: 4 - protein, 149 aa, synthetic construct
  source 1..149: note = C63S/M59R mutant CSF1 (33-181)
EEVSEYCSHMIGSGHLQSLQRLIDSQRETSSQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 5 - protein, 164 aa, synthetic construct
  source 1..164: note = C63S/M59R mutant CSF1 (33-196)
EEVSEYCSHMIGSGHLQSLQRLIDSQRETSSQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKA

SEQ ID NO: 6 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 7 - protein, 227 aa, Homo sapiens
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 8 - protein, 227 aa, synthetic construct
  source 1..227: note = R221K mutant granzyme B (21-247)
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGKNNGMPPRACTKVSSFVHWIKKTMKRY

SEQ ID NO: 9 - protein, 227 aa, synthetic construct
  source 1..227: note = note = R116A/R120A/R122A/K241A/K242A/K245A/R246A mutant granzyme B(21-247)
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLAKAKATAEAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIAATMAAY

SEQ ID NO: 10 - protein, 8 aa, synthetic construct
  source 1..8: note = FLAG-tag
DYKDDDDK

SEQ ID NO: 11 - protein, 25 aa, synthetic construct
  source 1..25: note = Exemplary fusogenic peptide that forms an alpha helix motif
ALEALAEALEALAEALEALAEAAAA

SEQ ID NO: 12 - protein, 19 aa, synthetic construct
  source 1..19: note = Exemplary leader sequence
MGWSCIILFLVATATGVHS

SEQ ID NO: 13 - protein, 5 aa, synthetic construct
  source 1..5: note = Sequence comprised in an exemplary linker
GGGGS

SEQ ID NO: 14 - protein, 18 aa, synthetic construct
  source 1..18: note = Exemplary cleavable linker
GGGGSLRMKLPKPGGGGS

SEQ ID NO: 15 - protein, 423 aa, synthetic construct
  source 1..423: note = GzmB-CSF1-FL, a fusion protein of the Examples
MGWSCIILFLVATATGVHSDYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 16 - protein, 449 aa, synthetic construct
  source 1..449: note = GzmB-CSF1-FLFP, a fusion protein of the Examples.
MGWSCIILFLVATATGVHSDYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQAALEALAEALEALAEALEALAEAAAA

SEQ ID NO: 17 - protein, 421 aa, synthetic construct
  source 1..421: note = GzmB-CSF1-CL, a fusion protein of the Examples.
MGWSCIILFLVATATGVHSDYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSLRMKLPKPGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 18 - protein, 464 aa, synthetic construct
  source 1..464: note = GzmB-CSF1-FLFPlong, a fusion protein of the Examples
MGWSCIILFLVATATGVHSDYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAAALEALAEALEALAEALEALAEAAAA

SEQ ID NO: 19 - protein, 415 aa, synthetic construct
  source 1..415: note = GzmB-CSF1, a fusion protein of the Examples
MGWSCIILFLVATATGVHSIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 20 - protein, 415 aa, synthetic construct; identical to SEQ ID NO: 19
  source 1..415: note = GzmB-CSF1-FL minus FLAG-tag

SEQ ID NO: 21 - protein, 441 aa, synthetic construct
  source 1..441: note = GzmB-CSF1-FLFP minus FLAG-tag
MGWSCIILFLVATATGVHSIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQAALEALAEALEALAEALEALAEAAAA

SEQ ID NO: 22 - protein, 413 aa, synthetic construct
  source 1..413: note = GzmB-CSF1-CL minus FLAG-tag
MGWSCIILFLVATATGVHSIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSLRMKLPKPGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 23 - protein, 456 aa, synthetic construct
  source 1..456: note = GzmB-CSF1-FLFPlong minus FLAG-tag
MGWSCIILFLVATATGVHSIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAAALEALAEALEALAEALEALAEAAAA

SEQ ID NO: 24 - protein, 404 aa, synthetic construct
  source 1..404: note = GzmB-CSF1-FL minus leader
DYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 25 - protein, 430 aa, synthetic construct
  source 1..430: note = GzmB-CSF1-FLFP minus leader
DYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQAALEALAEALEALAEALEALAEAAAA

SEQ ID NO: 26 - protein, 402 aa, synthetic construct
  source 1..402: note = GzmB-CSF1-CL minus leader
DYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSLRMKLPKPGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 27 - protein, 445 aa, synthetic construct
  source 1..445: note = GzmB-CSF1-FLFPlong minus leader
DYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAAALEALAEALEALAEALEALAEAAAA

SEQ ID NO: 28 - protein, 396 aa, synthetic construct
  source 1..396: note = GzmB-CSF1 minus leader
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 29 - protein, 396 aa, synthetic construct; identical to SEQ ID NO: 28
  source 1..396: note = GzmB-CSF1-FL minus leader and FLAG-tag

SEQ ID NO: 30 - protein, 422 aa, synthetic construct
  source 1..422: note = GzmB-CSF1-FLFP minus leader and FLAG-tag
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQAALEALAEALEALAEALEALAEAAAA

SEQ ID NO: 31 - protein, 394 aa, synthetic construct
  source 1..394: note = GzmB-CSF1-CL minus leader and FLAG-tag
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSLRMKLPKPGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 32 - protein, 437 aa, synthetic construct
  source 1..437: note = GzmB-CSF1-FLFPlong minus leader and FLAG-tag
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSGGGGSGGGGSGGGGSEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAAALEALAEALEALAEALEALAEAAAA

SEQ ID NO: 33 - protein, 8 aa, synthetic construct
  source 1..8: note = Exemplary cleavable linker
LRMKLPKP

SEQ ID NO: 34 - protein, 12 aa, synthetic construct
  source 1..12: note = Exemplary cleavable linke
GGGSLRMKLPKP

SEQ ID NO: 35 - protein, 4 aa, synthetic construct
  source 1..4: note = amino acid sequence comprised in an exemplary linker
GGGS

SEQ ID NO: 36 - protein, 415 aa, synthetic construct
  source 1..415: note = amino acid sequence of GzmB-CSF1-CL containing alternative cleavable linker
MGWSCIILFLVATATGVHSDYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGSLRMKLPKPEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 37 - protein, 407 aa, synthetic construct
  source 1..407: note = amino acid sequence of GzmB-CSF1-CL containing alternative cleavable linker, minus FLAG-tag
MGWSCIILFLVATATGVHSIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGSLRMKLPKPEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 38 - protein, 396 aa, synthetic construct
  source 1..396: note = amino acid sequence of GzmB-CSF1-CL containing alternative cleavable linker, minus leader
DYKDDDDKIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGSLRMKLPKPEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ

SEQ ID NO: 39 - protein, 388 aa, synthetic construct
  source 1..388: note = amino acid sequence of GzmB-CSF1-CL containing alternative cleavable linker, minus leader and FLAG-tag
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGSLRMKLPKPEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQ